(19) United States
(12) Patent Application Publication
(10) Pub. No.: US 2019/0352511 A1
(43) Pub. Date: Nov. 21, 2019

(54) LANTHANIDE COMPLEXES COMPRISING DENDRIMERS

(71) Applicants: UNIVERSITE D'ORLEANS, Orleans (FR); Centre national de la recherche scientifique, Paris (FR)

(72) Inventors: Stéphane PETOUD, Paris (FR); Svetlana ELISEEVA, Saint Jean Le Blanc (FR); Ivana MARTINIC, Orleans (FR); Virginie PLACIDE, Orleans (FR); Franck SUZENET, La Chapelle Saint Mesmin (FR); Régis DELATOUCHE, Orleans (FR); Joan LECLERC, Amboise (FR); Alexandra FOUCALT-COLLET, San Diego, CA (US); Guillaume COLLET, San Diego, CA (US)

(21) Appl. No.: 16/477,103

(22) PCT Filed: Jan. 10, 2018
(86) PCT No.: PCT/EP2018/050510

§ 371 Date: Jul. 10, 2019

(30) Foreign Application Priority Data
Jan. 11, 2017 (FR) 17 50245

(51) Int. Cl. C09B 69/10; C08G 83/00; A61K 41/00; G01N 21/64; G01N 15/14

(52) U.S. Cl. CPC C09B 69/107; C08G 83/003; G01N 15/1429; G01N 21/6458; G01N 21/6428; A61K 41/0071

(57) ABSTRACT

The present invention relates to a complex comprising at least one dendrimer and at least one lanthanide, wherein the dendrimer comprises a unit of formula (I) below:

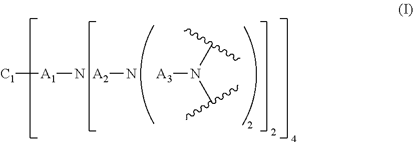

in which:
C₁ is a valence group 4 of formula >N—CH₂—CH₂—N<;
A₁, A₂ and A₃ are groups of formula —(CH₂)₂—C(O)—NH—(CH₂)₂—; the unit of formula (I) being connected covalently to at least one antenna which absorbs at a wavelength ranging from 500 nm to 900 nm.

The present invention relates to lanthanide complexes based on dendrimers. In particular, the subject of the present invention is dendrimeric compounds, grafted to an antenna, capable of complexing with lanthanides.

The methods related to fluorescence (microscopy and macroscopy) have the advantages of being very sensitive, to allow real-time measurements that are little dangerous for biological media (because of the small quantity of imaging agents required subject to using suitable excitation wavelengths) and very accessible (in terms of manufacturing and use costs), experimental time, mobility and degree of specialization of users.

The vast majority of commercial organic fluorophores are highly photosensitive and degrade in the presence of excitation light.

Fluorescent reporters based on semiconductor nanocrystals are another range of optical imaging agent choices. Another limiting factor is the high toxicity of the metals that make up these nanoparticles (cadmium, tellurium and selenium) in the case where the latter dissociate.

The family of lanthanides includes 14 elements with extremely interesting and unique optical properties, characterized by narrow and precise emission bands, ranging from visible to near infrared (>1200 nm). Each lanthanide has distinct and identifiable spectral properties. It is thus possible to identify, on the basis of the same technology, a whole range of different wavelengths simply by choosing the nature of the lanthanide to be incorporated in the molecule. These emission bands are much narrower than organic fluorophores and fluorescent nanoparticles (quantum dots), which allows for better spectral discrimination and multiplexed assays. In addition, the position (in nm) of these emission bands does not vary according to the environment (cell, pH, temperatures, hydrophilic/hydrophobic sites . . . ) which facilitates their detection and minimizes the adaptation of the equipment (unique filter for a given lanthanide).

Despite the strong demand from biology researchers and physicians, to date there are no fluorescent probes compatible with biological applications and operating in lower energy conversion based on lanthanides which are ideally excitable and emitters above 600 nm, corresponding to the biological window. Overall, the current near-infrared probes are of an organic nature, while the commercial probes are few and suffer from limitations such as the tendency towards photobleaching and restricted Stokes displacements.

The object of the present invention is therefore to provide a polymetallic lanthanide dendrimeric complex emitting in the near infrared and capable of being excited in the near infrared.

Another object of the invention is to provide an absorbing and emitting luminescent system in the near infrared and to observe various biological systems without destroying them or interfering with their operation. Another object of the invention is to provide a luminescent system for limiting spurious fluorescence/luminescence signals generated by biological materials (autofluorescence).

Thus, the present invention relates to a complex comprising at least one dendrimer (D) and at least one lanthanide (Ln), in which the dendrimer (D) comprises a unit of formula (I) below:

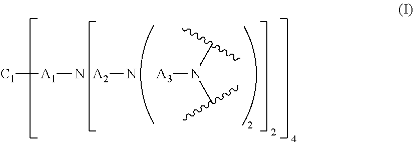

in which:
C₁ is a valence group 4 of formula >N—CH₂—CH₂—N<;
A₂ and A₃ are groups of formula —(CH₂)₂—C(O)—NH—(CH₂)₂—;

said unit of formula (I) being covalently connected to at least one antenna which absorbs at a wavelength ranging from 500 nm to 900 nm.

Luminescent compounds containing lanthanides have, among other advantages, a spectral specificity in the visible and near infrared spectral discrimination by their narrow emission bands specific to the nature of each lanthanide. In order to obtain a good luminescence intensity, it is important to introduce functional groups on the molecule that make it capable of absorbing a large amount of light radiation and to transfer the resulting energy onto the luminescent lanthanide in order to obtain a luminescence radiation by return of the lanthanide to the fundamental state.

Near infrared offers many unique advantages. It makes it possible to dispense with the auto-fluorescence of the tissues, and thus makes it possible to improve the signal-to-noise ratio and thus the detection sensitivity. Moreover, the biological tissues do not, or only slightly, absorb between 640 nm and 1100 nm (window of biological transparency), which allows excitation of the probes in depth and non-invasive biological observation (diagnosis and research).

In addition, the lanthanide complexes are photostable. This photostability property is crucial in allowing the imaging agent to be excited i) over long experimental times and/or ii) during successive experiments and/or iii) by powerful excitation sources (lasers for confocal microscopy, for example). A larger amount of photons may thus be collected without disturbing or damaging the functioning of the biological system to be studied (it is important to remember here that the excitation wavelength (>650 nm) interacts extremely weakly with fluids and tissues. and increases the intensity and quality of the signal collected.

The structure of the dendrimers makes it possible to group on the same molecule a large density of lanthanides and antennas making it possible to increase the quantity of photons emitted per unit volume, which increases the intensity of the signal per molecule and therefore the sensitivity of the detection.

The present invention thus relates to an entity obtained by complexation between a dendrimer (D) as defined above and at least one lanthanide. The complexes thus obtained are luminescent molecules.

The lanthanides are encapsulated within the dendrimer due to the presence of oxygen and nitrogen atoms. The arrangement of the dendrimer branches around the lanthanides partially protects them from direct interactions with the solvent and water molecules in particular.

Preferably, the complexes according to the invention are obtained by placing a dendrimer (D) in contact with a solution of lanthanides. The dendrimer (D) is dissolved, for example, in a solution of DMSO and a solution of lanthanide salt is added to the solution containing the compound (D), in particular in an eight to one ratio. According to one embodiment, the reaction mixture was mixed and the resulting dendrimer-lanthanide conjugate was isolated by dialysis.

According to one embodiment, in the compound (D), the unit of formula (I) is connected via at least one arm to at least one antenna as defined above.

Preferably, of the 16 peripheral nitrogen atoms in the formula (I), at least one is covalently connected to at least one antenna via an arm.

According to the invention, the term “antenna” designates an entity capable of absorbing a large amount of excitation light to transfer the energy corresponding to the lanthanides and/or to emit directly by fluorescence.

Preferably, the antenna is selected from the group consisting of anthraquinones, cyanines, especially cyanines 5, cyanines 5.5 and cyanines 7, aza-BODIPY, perylenediimides, porphyrins, phenothiazine salts and their derivatives.

Among the antennas, it is also possible to use compounds of the “IR dyes” type well known to those skilled in the art. Among these antennas, may be mentioned for example the following compounds:

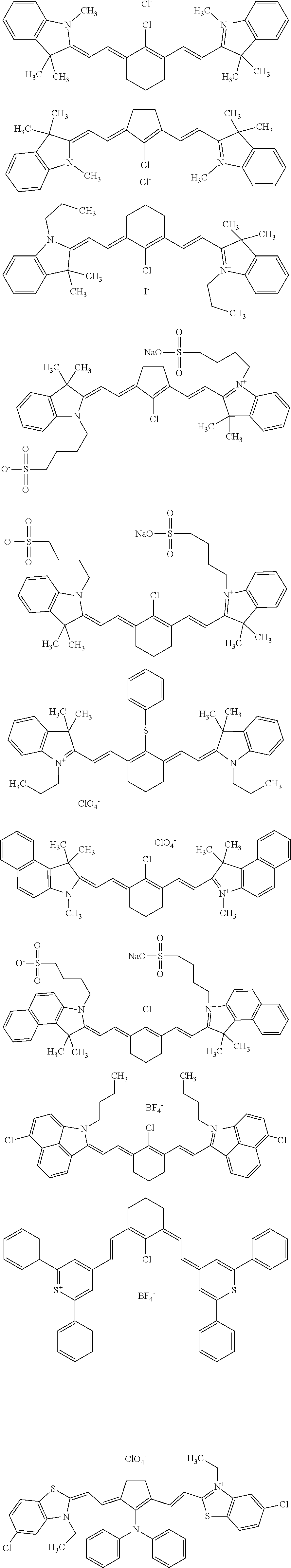

These antennas are fixed on the dendrimers via chlorine by substitution and thus in the final form there is no chlorine but a C, N, S and O.

According to the invention, the term “arm” refers to an entity for covalently connecting the pattern of formula (I) and the antenna.

According to one embodiment, the antenna is connected to the unit of formula (I) in a covalent manner via at least one arm corresponding to the following formula (II):

-A₄-X-[A₅-Z]ᵢ-((A₆)ⱼ-Y)ₖ- (II)

in which:
A₄ is a group of formula —(CH₂)₂—X′—(CH₂)₂—, X′ representing a —C(O)—NH— group or a —NH—C(O)— group, and being preferably a group of formula —(CH₂)₂—C(O)—NH—(CH₂)₂—;
X is a group of formula —NH—C(O)—;
i is an integer between 1 and 3;
j is 0 or 1;
k is 0 or 1;
A₅ and A₆ are chosen, independently of one another, from linear or branched (cyclo) alkylene radicals comprising from 1 to 12 carbon atoms;
Z is selected from: —O—, —NH—, —S—, amide, ester, triazole, amine, ether, thioether, urea, thiourea, imine, oxyme, hydrazone, sulfonamide, carbamate, amidine, phosphoramidate, disulfide, and sulfonyl; and
Y is selected from: —O—, —NH—, —S—, alkylene, amide, ester, triazole, amine, ether, thioether, urea, thiourea, imine, oxyme, hydrazone, sulfonamide, carbamate, amidine, phosphoramidate, disulfide, and sulfonyl.

In the formula (II) as defined above, it is through the radical A₄ that the arm binds to the dendrimer (in particular the unit of formula (I) above) and it is via Y, or Z when k=0, that the arm binds to the antenna.

According to a preferred embodiment, the dendrimer has the following formula:

C₁-{A₁-N[A₂-N(A₃-N(A₄-R′)₂)₂]₂}₄ (III)

in which:
C₁ is a valence group 4 of formula >N—CH₂—CH₂—N<;
A₁, A₂, A₃ and A₄ are groups of formula —(CH₂)₂—C(O)—NH—(CH₂)₂—;
the radicals R′ are chosen, independently of one another, from the group consisting of:
   groups of formula (1) below:

—NH—C(O)-[A₅-Z]ᵢ-((A₆)ⱼ-Y)ₖ-L
i, j, k, A₅, A₆, Z and Y being as defined in formula (II),
L representing an antenna selected from the group consisting of anthraquinones, cyanines, aza-BODIPY, perylenediimides, porphyrins, phenothiazine salts, and their derivatives;
   groups of formula (2) below:

—NH—C(O)-[A₅-Z]ᵢ-((A₆)ⱼ-Y)ₖ-L′
j, k, A₅, A₆, Z and Y being as defined in formula (II),
L′ representing a water-solubilising group or a targeting group; and
   groups of formula (3)

—NH—C(O)-A₅-L″
A₅ being as defined in formula (II), and
L″ representing a function which may be involved in a bioconjugation reaction, and preferably being an alkyne group;
   at least one of the groups R′ corresponding to the formula (1).

According to the invention, the term “water-solubilising group” designates a chemical entity making it possible to increase the solubility of the probe in aqueous media.

Among the water-solubilising groups, may be mentioned, for example, phosphates, sulphonates, sugars and PEG chains.

According to the invention, the term “targeting group” refers to a molecule, biological or not, capable of recognizing and/or binding a specific biological site.

Targeting groups include, for example, antibodies, proteins, peptides, carbohydrates, lipids, polysaccharides, fatty acids, amino acids, deoxyribonucleic acids, ribonucleic acids, oligonucleotides, medicaments, and ligands.

Among the water-solubilising groups and the targeting groups, may be mentioned the following groups:

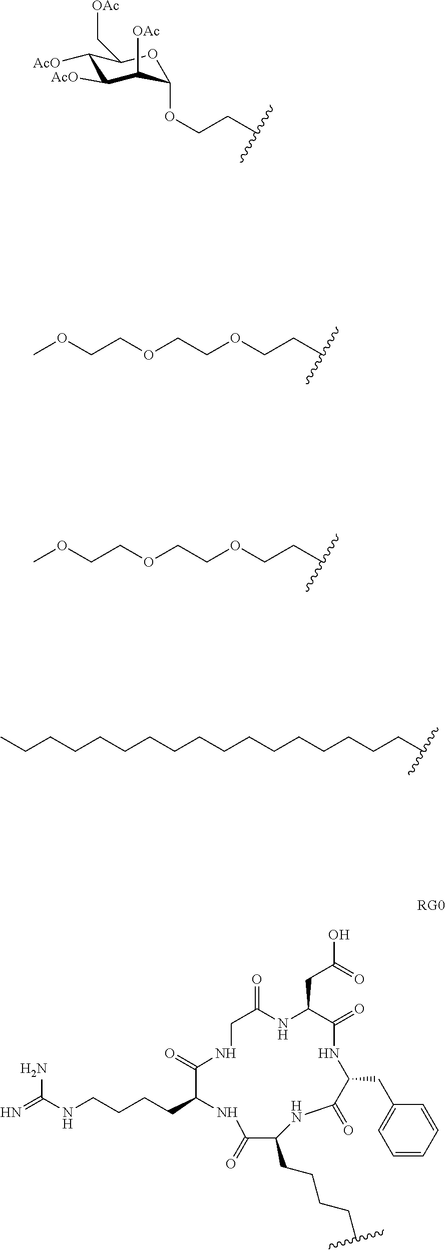

Formula (II) above contains 32 peripheral R′ groups, of which at least one comprises an antenna connected to the dendrimer via an arm. Thus, the dendrimers according to the invention may comprise at least one arm through which at least one antenna is linked.

Thus, as indicated above, at least one of the R′ groups has the formula (1) above.

According to one embodiment, the dendrimer is a generation 4 dendrimer of the following formula (III′):

C₁-{A₁N[A₂-N(A₃-N(A₄-N[(CH₂)₂—C(O)—NH—(CH₂)₂—R′]₂)₂)₂]₂}₄

R′ being as defined above.

Preferably, in formula (1), k=0.

Preferably, in formula (1), k=0 and i=1.

According to one embodiment, in the formula (II), at least one of the R′ groups corresponds to the following formula (1′):

—NH—C(O)-A₅-Z-L

A₅ and Z being as defined in formula (II), and

L being as defined above.

According to one embodiment, in the formula (II), all the identical R′ groups correspond to the formula (1′).

Preferably, in the formula (1′) above, A₅ is chosen from alkylene radicals, linear or branched, comprising from 1 to 4 carbon atoms.

Preferably, in formula (1′) above, Z is selected from the group consisting of —O—, —NH—, —S—, —C(═O)—O—, —O—C(═O)—, —NH—C(═O)—, —N(Alk)-C(═O)—, —C(═O)—NH— et —C(═O)—N(Alk)- groups, Alk representing an alkyl group having from 1 to 6 carbon atoms.

According to one embodiment, in the formula (II), at least one of the R′ groups corresponds to the following formula (1″):

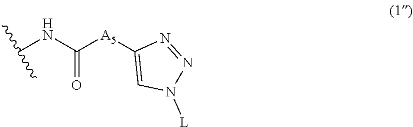

A₅ and L being as defined above.

Preferably, in formula (1″) above, A₅ is chosen from alkylene radicals, linear or branched, comprising from 1 to 4 carbon atoms.

According to one embodiment, in the formula (II), all the identical R′ groups correspond to the formula (1″).

According to one embodiment, in formula (II), at least one of the R′ groups corresponds to the following formula (1′″):

—NH—C(O)-A₅-O-L

A₅ and L being as defined above.

Preferably, in the formula (1′″) above, A₅ is chosen from alkylene radicals, linear or branched, comprising from 1 to 4 carbon atoms.

According to one embodiment, in the formula (II), all the identical R′ groups correspond to the formula (1′″).

According to one embodiment, the antenna responds to one of the following formulas:

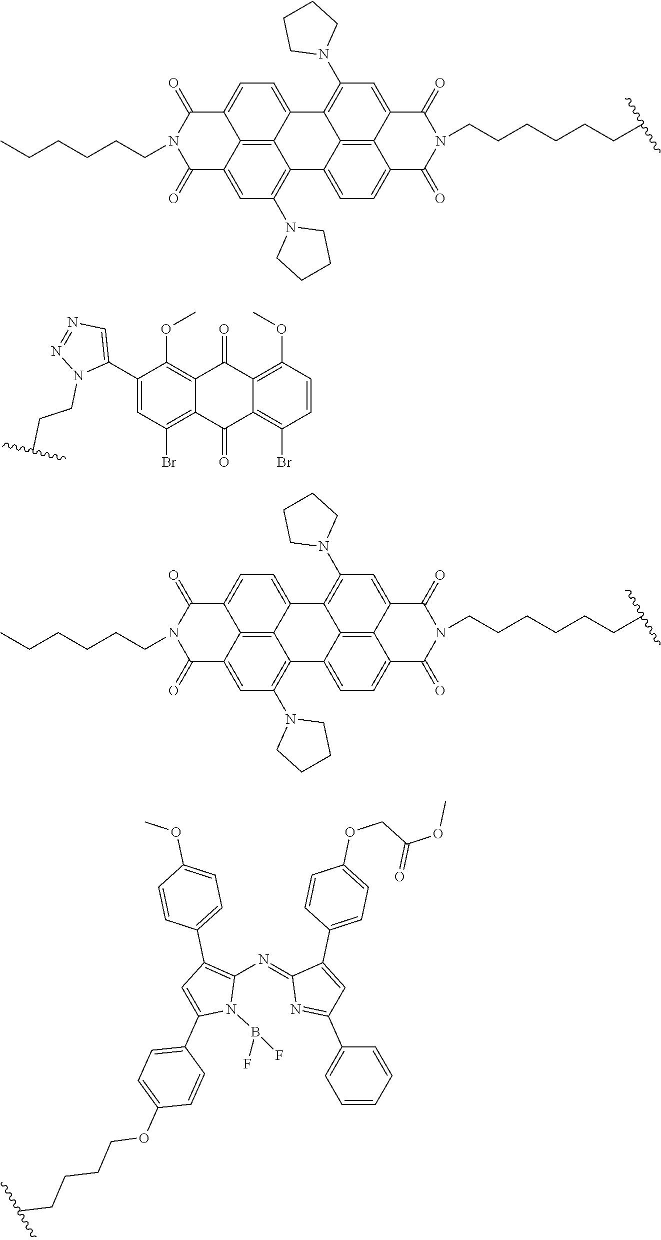

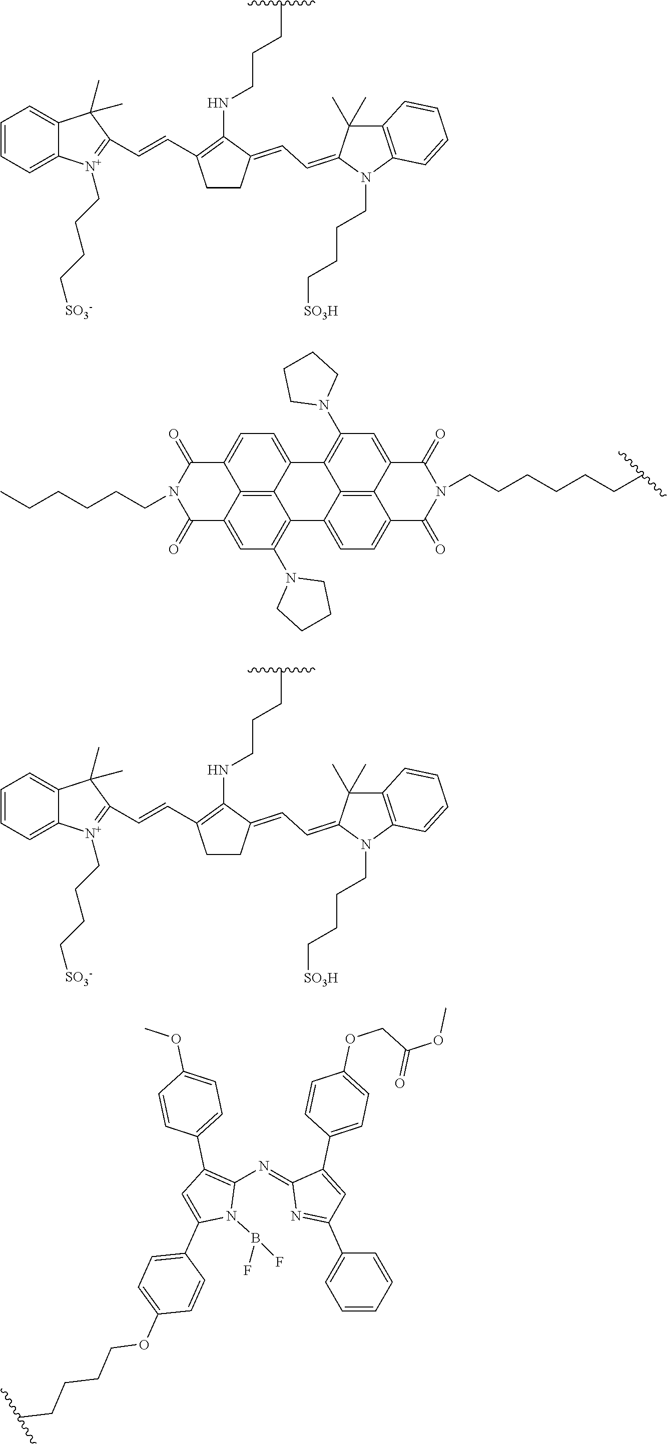

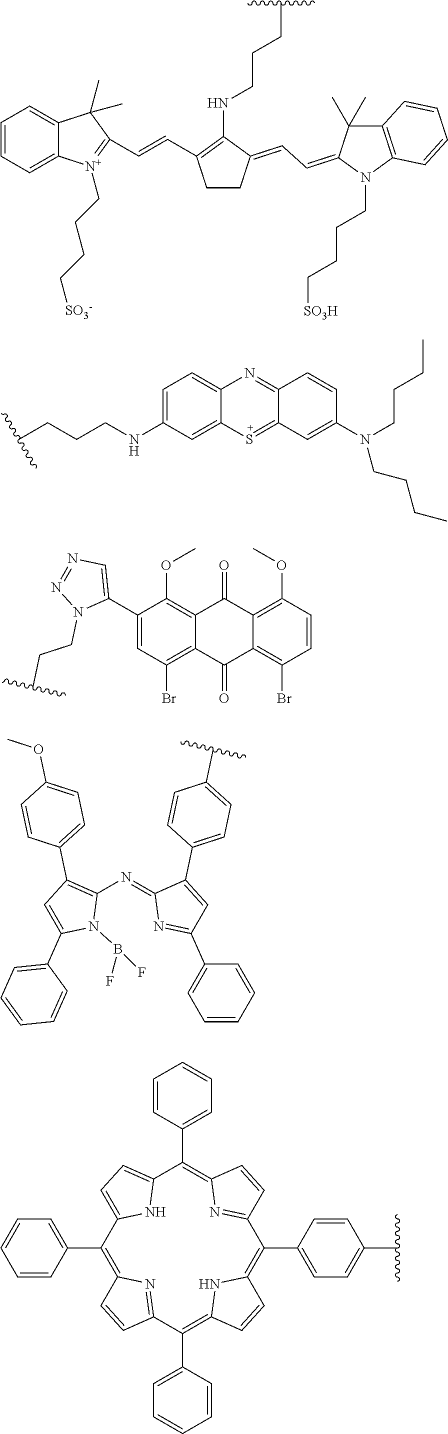

When the antenna meets the formula

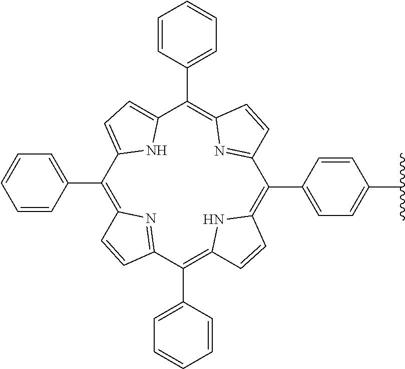 [SMILES: *c1ccc(-c2c3nc(c(-c4ccccc4)c4ccc([nH]4)c(-c4ccccc4)c4nc(c(-c5ccccc5)c5ccc2[nH]5)C=C4)C=C3)cc1]

this may contain a metal, chosen in particular from Ag, Al, As, Au, Cd, Co, Cu, Fe, Ir, Mg, Mn, Ni, Os, Pd, Pt, Rh, Ru, Sb, Sn, V, Zn, La, Ce, Pr, Nd, Sm, Eu, Gd, Tb, Dy, Ho, Er, Tm, Yb, and Lu.

Preferably, the dendrimer (D) according to the invention has the following formula:

in which L₂ is one of the following antennas:

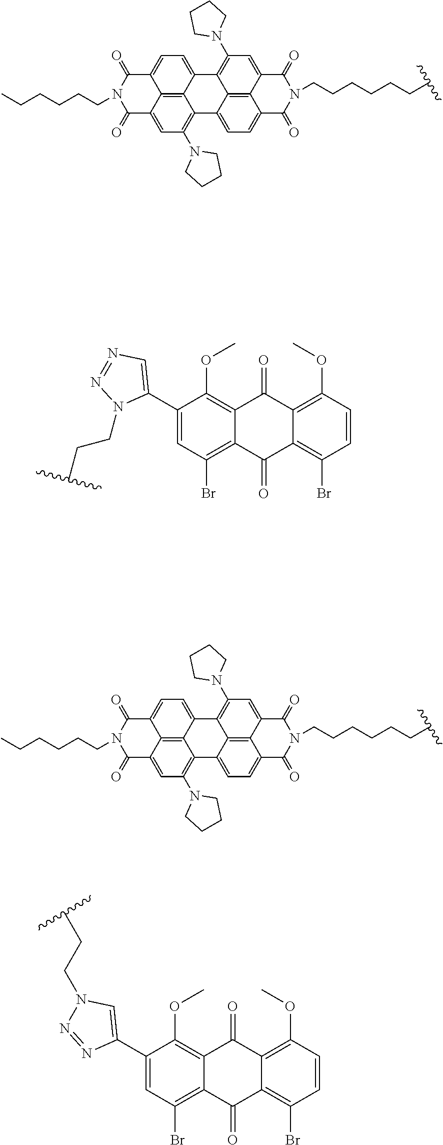 [SMILES: *CCCCCCN1C(=O)c2ccc3c4c(N5CCCC5)cc5c6c(ccc(c7c(N8CCCC8)cc(c2c37)C1=O)c64)C(=O)N(CCCCCC)C5=O ; *CCn1nncc1-c1cc(Br)c2c(c1OC)C(=O)c1c(OC)ccc(Br)c1C2=O ; *CCCCCCN1C(=O)c2ccc3c4c(N5CCCC5)cc5c6c(ccc(c7c(N8CCCC8)cc(c2c37)C1=O)c64)C(=O)N(CCCCCC)C5=O ; *CCn1cc(-c2cc(Br)c3c(c2OC)C(=O)c2c(OC)ccc(Br)c2C3=O)nn1]

Preferably, the dendrimer (D) according to the invention has the following formula:

in which R corresponds to the following formula:

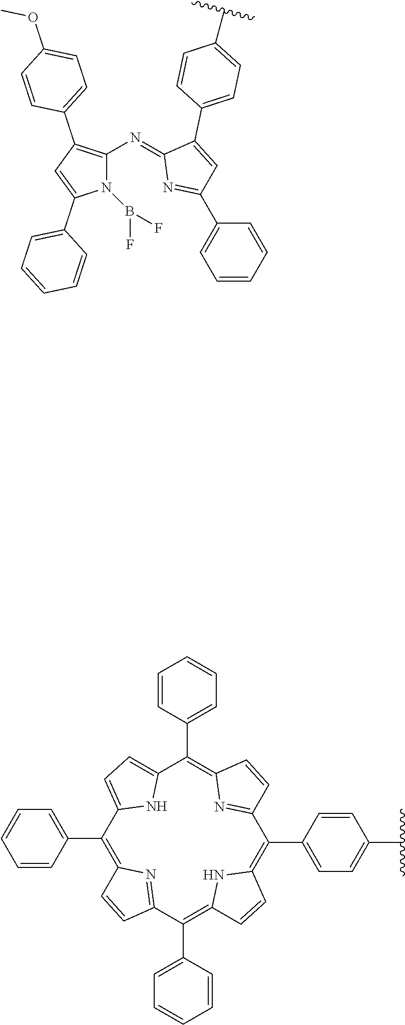 [SMILES: *c1ccc(C2=CC(c3ccccc3)=N/C2=N\c2c(-c3ccc(OC)cc3)cc(-c3ccccc3)n2B(F)F)cc1 ; *c1ccc(-c2c3nc(c(-c4ccccc4)c4ccc([nH]4)c(-c4ccccc4)c4nc(c(-c5ccccc5)c5ccc2[nH]5)C=C4)C=C3)cc1]

Preferably, the dendrimer (D) according to the invention has the following formula:

in which L₂ is one of the following antennas:

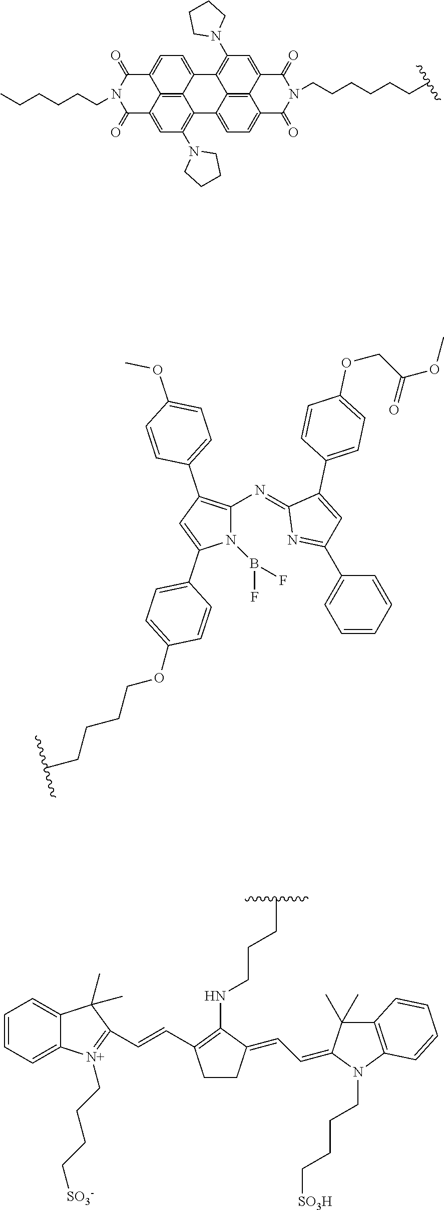

Preferably, the dendrimer (D) according to the invention has the following formula:

in which:
L₂ corresponds to one of the following formulas:

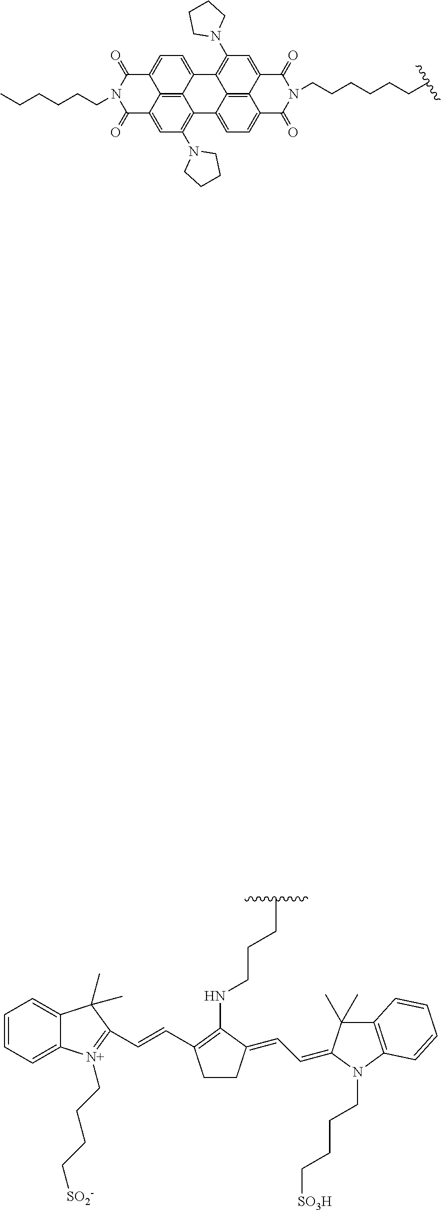

R₂ corresponds to one of the following formulas:

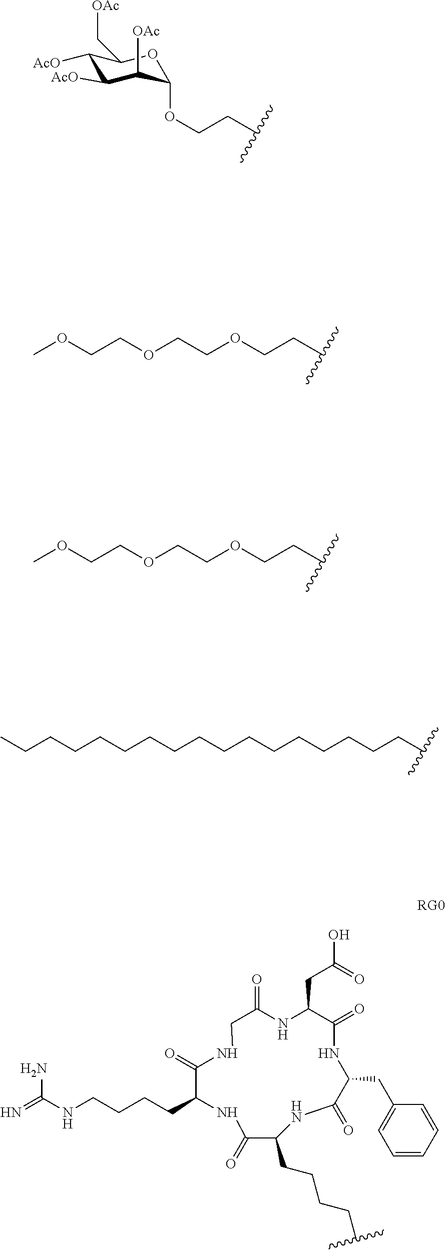

According to one embodiment, in the complexes according to the invention, the lanthanide is chosen from the group consisting of Yb, Nd, Ho, Tm, Sm, Dy, Eu, Pr and Er.

The present invention also relates to a conjugate comprising a biological molecule and a complex as defined above, wherein said complex is linked to the biological molecule via a linker, said biological molecule being chosen from the group consisting of antibodies, proteins, peptides, carbohydrates, lipids, polysaccharides, fatty acids, amino acids, deoxyribonucleic acids, ribonucleic acids, oligonucleotides, drugs, and ligands.

The present invention also relates to the use of a complex as defined above as a fluorescent chromophore.

According to the invention, the term “fluorescent chromophore” refers to a molecule that can re-emit light after excitation with a quantum yield greater than 10⁻⁶ (10⁻⁴%).

The complexes according to the invention may especially be used in the field of cell imaging, veterinary imaging, blood tests, biopsies, histological sectional analyzes, high throughput screening assays, bioanalytical assays. plates 96, 396 and 1536 wells or assisted (guided) surgery by imaging.

The present invention also relates to the use of a complex as defined above as a photodynamic therapy (PDT) agent.

### DESCRIPTION OF THE FIGURES

FIG. 1 shows the ¹H NMR spectrum (DMSO, TFA, 40° C.) of the G3P-(TPP)₃₂ dendrimer.

FIG. 2 shows the ¹H NMR spectrum (DMSO, room temperature, water suspension) of the G3P-(TPP)₃₂ dendrimer.

FIG. 3 shows the absorption spectra of a solution of compound 9 at a concentration of 10 μM and (Ln₈-) G3P-(Bodipy1)ₙ (n=16,32; Ln=Yb³⁺, Nd³⁺) measured at room temperature in the DMSO. For the sake of comparison, the absorption spectrum of compound 9 is multiplied by a factor corresponding to the number of chromophores attached to the branches of the dendrimer, for example, by 16 for (Ln₈-) G3P-(Bodipy1)₁₆ and by 32 for (Ln₈-) G3P-(Bodipy1)₃₂.

FIG. 4 represents the excitation spectra (λₑₘ=750 nm, left graph) and emission (λₑₓ=650 nm, right graph) spectra normalized of a solution of concentration of 10 μM of compound 9 and (Ln₈-)G3P-(Bodipy1)ₙ (n=16, 32; Ln=Yb³⁺, Nd³⁺) measured in DMSO at room temperature.

FIG. 5 shows the results of a photobleaching experiment for compound 9 and (Ln₈-) G3P-(Bodipy1)ₙ (n=16, 32; Ln=Yb³⁺, Nd³⁺). The experiments were carried out for a solution at 10 μM in DMSO and under uninterrupted illumination at 670 nm, the emission being collected at 735 nm.

FIG. 6 shows the results of the Alamar Blue cytotoxicity tests carried out for different concentrations of chromophores (compound 9) and dendrimers (Ln₈-)G3P-(Bodipy1)ₙ (n=16, 32; Ln=Yb³⁺, Nd³⁺) after 24 h incubation on HeLa cells.

FIG. 7 shows the confocal microscopy imaging results. HeLa cells after 30 min of incubation with a solution of 1.5 μM compound 9 and dendrimer (Ln₈-)G3P-(Bodipy1)ₙ (n=16, 32; Ln=Yb³⁺, Nd³⁺). The emission of aza-BODIPY (λₑₓ: 633 nm, λₑₘ: 650-800 nm) was observed under excitation at 633 nm (laser power at 6%). (Top) Yb₈-G3P-(Bodipy1)₁₆ (A), Nd₈-G3P-(Bodipy1)₁₆ (B), G3P-(Bodipy1)₁₆ (C), Compound 9 (D) and (bottom) Yb₈-G3P-(Bodipy1)₃₂ (A), Nd₈-G3P-(Bodipy1)₃₂ (B), G3P-(Bodipy1)₃₂ (C), untreated cells (D). The images are taken in the plane of the cellular nucleus. The white arrows indicate the specific localization of the dendrimers in the lysosomes and the yellow arrows indicate the filopods. 63× magnification lens.

FIG. 8 shows the results of the study of cellular internalization by flow cytometry. HeLa cells were incubated for 30 min with a 1.5 μM concentration solution of chromophores and dendrimers (Ln₈-)G3P-(Bodipy1)ₙ (n=16, 32 ; Ln=Yb³⁺, Nd³⁺) as control. In order to block the active and passive transport pathways, HeLa cells were treated with NaN₃ (active internalization inhibitor) or pre-incubated at 4° C. for 30 min (inhibition of active and passive pathways) before incubation with the dendrimers.

FIG. 9 shows epifluorescence microscopy imaging results on HeLa cells after 30 minutes of incubation with a 1.5 μM solution of compound 9 and dendrimers (Ln₈-)G3P-(Bodipy1)ₙ (n=16, 32; Ln=Yb³⁺, Nd³⁺). The signal emitted by the aza-Bodipy chromophore was collected (λₑₓ: 655 nm±40 nm, λₑₘ: long pass filter 785 nm, 8 s exposure). (Top) Yb₈-G3P-(Bodipy1)₁₆ (A), Nd₈-G3P-(Bodipy1)₁₆ (B), G3P-(Bodipy1)₁₆ (C), Compound 9 (D) and (bottom) Yb₈-G3P-(Bodipy1)₃₂ (A), Nd₈-G3P-(Bodipy1)₃₂ (B), G3P-(Bodipy1)₃₂ (C), untreated cells (D).

FIG. 10 shows absorption and emission spectra of G3P-(Alcyne)ₓ-(Cyanine)_{y} (x=22-24, y=8-10) in DMSO at room temperature.

FIG. 11 shows the results of the photostability tests of the (Ln₈-)G3P-(Alcyne)ₓ-(Cyanine)_{y} complexes (x=22-24, y=8-10, Ln=Yb, Nd) under excitation at 665 nm (DMSO, room temperature, λₑₘ=725 nm).

FIG. 12 shows the results of epifluorescence microscopy imaging on HeLa cells. (Top) After 3 h of incubation with a 2.5 μM solution of (Nd₈-)G3P-(Alcyne)ₓ-(Cyanine)_{y} (x=22-24, y=8-10) and (bottom) non processed. A: image obtained in white light. B: luminescence image (λₑₘ: 655 bandwidth 40 nm, λₑₘ: 750 bandwidth 50 nm, exposure time of (seconds) C: image resulting from the fusion of A and B.

FIG. 13 shows the absorption spectrum of Yb₈-G3P-(TPP)₃₂ (1.0 μM) in DMSO and in the DMSO/(Opti-MEM: FCS) mixture (2.2 μM) at 298 K.

FIG. 14 shows the visible emission spectrum of Yb₈-G3-(TPP)₃₂ (37 μM) in DMSO under excitation at 520 nm, 298K.

FIG. 15 shows the near-infrared emission spectra of Yb₈-G3-(TPP)₃₂ (37 μM) and G3-(TPP)₃₂ (37 μM) in DMSO under excitation at 520 nm, 298K.

FIG. 16 shows the absorption and excitation spectra (λₑₘ=977 nm) of the Yb₈-G3-(TPP)₃₂ complex in DMSO normalized to the intensity of the 650 nm band.

FIG. 17 shows the images obtained by epifluorescence microscopy of HeLa cells. (A) After 1 h and 30 min of incubation with a 1 μM solution of Yb-G3P-TPP dendrimer complex. (B) The same cells after 2 min of selected light exposure with a bandpass filter centered at 417 nm: vesicle formation. (C) cells not incubated with Yb₈-G3-(TPP)₃₂.

### EXAMPLES

### Example 1: Synthesis of Dendrimers (D) According to the Invention

### 1. Preparation of Antennas of aza-BODIPY Type

### 1.1. Preparation of tert-butylN-[2-[[2-[4-[(2Z)-[1-difluoroboranyl-3-(4-methoxyphenyl-5-phenyl-pyrrol-2-yl]imino-5-phenyl-pyrrol-3-yl]phenoxy]acetyl]amino]ethyl]carbamate (10)

This antenna (10) is obtained according to the reaction scheme below:

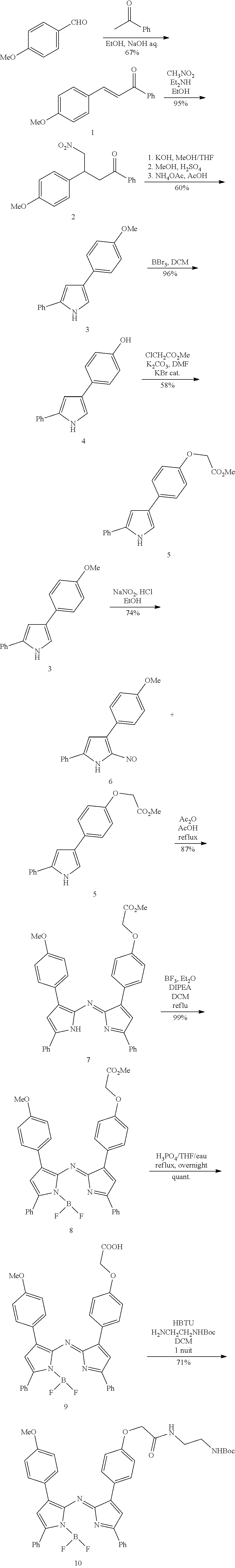

### Synthesis of (E)-3-(4-methoxyphenyl)-1-phenylprop-2-en-1-one (1)

8.94 ml of para-anisaldehyde (10 g, 73.45 mmol, 1 eq.) and 8.58 ml of acetophenone (8.825 g, 73.45 mmol, 1 eq.) are dissolved in a flask in 150 mL ethanol. The flask is immersed in an ice bath and 5.876 g of sodium hydroxide (149.9 mmol, 2 eq.) dissolved in 50 ml of water are added dropwise. The solution is allowed to warm to room temperature and stirred overnight. The next day, the flask is immersed in an ice bath and cold water is added to the mixture. A yellow precipitate forms and the mixture is sintered and washed with water. The precipitate is then recrystallized from ethanol yielding 11.698 g of chalcone 1 (49.1 mmol, 67%) as white crystals.

¹H NMR (250 MHz, Chloroform-d) δ8.06-7.97 (m, 2H), 7.79 (d, J=15.0 Hz, 1H), 7.64-7.59 (m, 2H), 7.58-7.45 (m, 3H), 7.42 (d, J=15.6 Hz, 1H), 6.98-6.91 (m, 2H), 3.86 (s), 3H).

### Synthesis of 3-(4-methoxyphenyl)-4-nitro-1-phenylbutane-1-one (2)

0.546 g (2.29 mmol, 1 eq.) of 0.621 mL of nitromethane (11.46 mmol, 5 eq.) and 1.185 mL of diethylamine (11.46 mmol, 5 eq.) are dissolved in a flask with 150 mL of methanol. The solution is heated to reflux overnight. After the reaction is complete, the solvent is evaporated in vacuo and the residue dissolved in dichloromethane. The solution is washed with a solution of 1M KHSO₄ then saturated NaCl. The organic phases are combined, dried over MgSO₄, filtered and concentrated in vacuo. The residue is purified by chromatography on silica with petroleum ether/ethyl acetate providing nitrobutanone 2 (0.654 g, 2.18 mmol, 95%) as a pale yellow viscous oil.

¹H NMR (250 MHz, Chloroform-d) δ7.96-7.88 (m, 2H), 7.63-7.53 (m, 1H), 7.51-7.41 (m, 2H), 7.25-7.16 (m, 2H), 6.91-6.81 (m, 2H), 4.80 (ddd, J=12.3, 6.7, 0.5 Hz, 1H), 4.65 (ddd, J=12.3, 7.9, 0.4 Hz, 1H), 4.18 (p, J=7.0 Hz, 1H), 3.78 (s, 3H), 3, 42 (dd, J=7.0, 2.1 Hz, 2H).

### Synthesis of 4-(4-methoxyphenyl)-2-phenyl-1H-pyrrole (3)

In a flask are dissolved 3.5 g of nitrobutanone 2 (11.69 mmol, 1 eq.) and 5 eq. of KOH in 100 mL of MeOH/THF (1:2). The solution is stirred at room temperature for one hour and then added dropwise to a solution of concentrated H₂SO₄ (2 mL/mmol) dissolved in 100 mL of MeOH at 0° C. After the addition is complete, the ice bath is removed and the solution stirred at room temperature for 1 h. The mixture is then poured into an Erlenmeyer flask containing water and ice and the solution is neutralized by adding a 4M sodium hydroxide solution. Once neutralized, the mixture is extracted with dichloromethane and the organic phase is dried over MgSO₄, filtered and the solvent evaporated under reduced pressure. The residue is dissolved in 100 mL glacial acetic acid and 5 eq. ammonium acetate are added. The solution is heated at reflux for one hour during which the color of the solution changes from yellow to deep blue. The solution is cooled to room temperature and the acetic acid evaporated under reduced pressure. The black solid is then dissolved in dichloromethane and the solution is washed several times with a saturated solution of sodium bicarbonate and brine. The organic phase is dried over MgSO₄, filtered and concentrated under reduced pressure. The residue is then dissolved in a minimum of dichloromethane and the petroleum ether is added slowly until a precipitate forms. The precipitate is filtered on Buchner and washed several times with petroleum ether. Pyrrole 3 is obtained with a yield of 60% (1742 g, 6.99 mmol) in the form of a slightly colored powder.

¹H NMR (250 MHz, DMSO-d₆) δ11.32 (s, 1H), 7.66 (dt, J=7.7, 1.1 Hz, 2H), 7.57-7.48 (m, 2H), 7.36 (dd, J=8.4, 7.0 Hz, 2H), 7.21 (dd, J=2.8, 1.7 Hz, 1H), 7.20-7.12 (m, 1H), 6.94-6.84 (m, 3H), 3.75 (s, 3H).

### Synthesis of 4-(5-phenyl-1H-pyrrol-3-yl)phenol (4)

0.546 g of pyrrole 3 (2.19 mmol, 1 eq.) are dissolved under argon in a flask in 100 ml of anhydrous dichloromethane. The solution is cooled to −78° C. and 5.48 ml of a solution of 1M BBr₃ in dichloromethane (5.48 mmol, 2.5 eq.) are slowly added. The reaction is stirred for 3 h at −78° C. and then overnight at room temperature. The next day, the solution is cooled to −78° C. and MeOH is added to the mixture. The solution is stirred for one hour then it is diluted in dichloromethane and washed with saturated NaCl. The organic phase is dried over MgSO₄, filtered and concentrated under reduced pressure. The residue is dissolved in a minimum of dichloromethane and the product is precipitated by slow addition of petroleum ether. The precipitate is filtered on Buchner and washed with petroleum ether yielding 0.495 g (2.10 mmol, 96%) of pyrrole 4 in the form of a slightly purplish white powder.

¹H NMR (250 MHz, DMSO-d₆) δ11.25 (s, 1H), 9.15 (s, 1H), 7.69-7.61 (m, 2H), 7.43-7.31 (m, 4H), 7.20-7.11 (m, 2H)), 6.81 (dd, J=2.7, 1.7 Hz, 1H), 6.77-6.69 (m, 2H).

¹H NMR (250 MHz, acetone-d₆) δ10.48 (s, 1H), 8.09 (s, 1H), 7.71-7.65 (m, 2H), 7.49-7.41 (m, 2H), 7.41-7.31 (m, 2H), 7.21-7.14 (m, 2H), 6.85 (dd, J=2.8, 1.7 Hz, 1H). 6.84 -6.80 (m, 2H).

¹³C NMR (63 MHz, Acetone) δ156.27, 134.16, 133.40, 129.58, 128.76, 126.92, 126.80, 126.55, 124.38, 116.24, 115 , 94, 104.14.

LRMS: calcd: 235.0997, measured [M+H]⁺: 236.1

IR (cm⁻¹): 3443, 3300, 1600, 1581, 1494, 1244, 1132, 921, 834, 804, 778, 751, 717, 690, 609.

Mp: 209° C.

### Synthesis of methyl 2-(4-(5-phenyl-1H-pyrrol-3-yl)phenoxy)acetate (5)

0.725 g of 4 (3.08 mmol, 1 eq.) in 60 ml of DMF are dissolved in a flask. 1.278 g of K₂CO₃ (9.24 mmol, 3 eq.), 1.08 ml of methyl chloroacetate (12.33 mmol, 4 eq.) and a catalytic amount of potassium bromide are added to the solution which is stirred at room temperature all night. The solution is then extracted with diethyl ether and washed three times with brine. The aqueous phase is then extracted three times with diethyl ether. The organic phases are combined and dried over MgSO₄, filtered and the solvent evaporated under vacuum. The residue is dissolved in a minimum of dichloromethane and petroleum ether is added until a precipitate forms. The precipitate is filtered through Buchner and washed with petroleum ether to give 0.553 g of 5 as an off-white powder.

¹H NMR (250 MHz, Chloroform-d) δ8.43 (s, 1H), 7.55-7.46 (m, 4H), 7.44-7.35 (m, 2H), 7.25-7.19 (m, 1H), 7.07 (dd, J=2.7, 1.7 Hz, 1H), 6.97-6.88 (m, 2H), 6.76 (dd, J=2.8, 1.7 Hz, 1H), 4.66 (s, 2H), 3.82 (s, 3H).

¹³C NMR (63 MHz, CDCl₃) δ169.57, 156.10, 133.02, 132.51, 129.56, 128.93, 126.45, 126.38, 126.11, 123.83, 114 , 98, 114.94, 103.86, 65.58, 52.25.

LRMS: Calcd: 307.1208, Measured [M+H]⁺: 308.4

IR (cm⁻¹): 3430, 2941, 1759, 1600, 1581, 1496, 1438, 1212, 1178, 1131, 1075, 834, 801, 774, 758, 719, 693.

Mp: 165° C.

### Synthesis of 3-(4-methoxyphenyl)-2-nitroso-5-phenyl-1H-pyrrole (6)

0.593 g of pyrrole 3 (2.38 mmol, 1 eq.) in 50 ml of ethanol and 0.48 ml of concentrated HCl (0.2 ml/mmol) are dissolved in a flask at room temperature. 0.189 g of sodium nitrite (2.74 mmol, 1.15 eq.) dissolved in water (concentration 0.6 mol/l) are added dropwise. The solution is stirred for 30 min and is then cooled to 0° C. A second portion of concentrated HCl (2.38 mL, 1 mL/mmol) is added. The solution is stirred for one hour then it is dissolved in dichloromethane and washed with brine. The organic phase is dried, filtered and concentrated under reduced pressure. The residue is dissolved in a minimum volume of ethanol and excess aqueous solution of sodium acetate and ice are added and the mixture is stirred for one hour. The solution is then extracted with dichloromethane and washed with brine. The organic phase is dried over MgSO₄, filtered and concentrated in vacuo. The residue is then dissolved in a minimum volume of dichloromethane and the product is precipitated by slow addition of petroleum ether. The solid is filtered on Buchner and washed with petroleum ether. 0.503 g of nitrosopyrrole 6 (1.81 mmol, 76%) are obtained in the form of a green powder.

¹H NMR (250 MHz, Chloroform-d) δ8.20-8.13 (m, 2H), 7.79 (dd, J=6.9, 3.0 Hz, 2H), 7.51 (dd, J=5.1, 1.8 Hz, 3H), 7.07 (s, 1H), 7.02 (d, J=8.9 Hz, 2H), 3.89 (s, 3H).

### Synthesis of (Z)-methyl 2-(4-(2-((3-(4-methoxyphenyl)-5-phenyl-1H-pyrrol-2-yl)imino)-5-phenyl-2H-pyrrol-3-yl)phenoxy)acetate (7)

20 g of glacial acetic acid (0.112 g of nitrosopyrrole 6 (0.40 mmol, 1 eq.), 0.124 g of pyrrole (0.40 mmol, 1 eq. 0.40 mL of acetic anhydride. The solution is stirred and heated at reflux for one hour during which the color changes to dark blue. The solution is then cooled and the solvent evaporated under reduced pressure. The residue is then dissolved in dichloromethane and the solution is washed with saturated NaHCO₃ and saturated NaCl. The organic phase is dried over MgSO₄, filtered and concentrated in vacuo. The residue is then dissolved in a minimum of dichloromethane and the petroleum ether is added until a precipitate forms which is filtered on Buchner and washed with petroleum ether. Azadipyrromethene 7 is obtained in the form of a dark blue powder (0.197 g, 0.35 mmol, 87%).

¹H NMR (250 MHz, Chloroform-d) δ8.05-7.98 (m, 4H), 7.93 (ddd, J=7.8, 6.4, 1.5 Hz, 4H), 7.58-7.44 (m, 7H), 7.11 (s, 1H), 7.09 (s, 1H), 6.96 (dd, J=8.9, 1.8 Hz, 4H), 4.71 (s, 2H), 3.90 (s, 3H), 3.85 (s, 3H).

IR (cm⁻¹): 3426, 2948, 1758, 1597, 1495, 1211, 1175, 1075, 1002, 902, 833, 801, 772, 759, 745, 718, 692, 637, 605.

Mp: 230° C.

HRMS (ESI): m/z calcd for [C36H30N3O4]: 568.223083, measured 568.222834 (−0.4 ppm)

### Synthesis of (Z)-methyl2-(4-(2((1-(difluoroboryl)-3-(4-methoxy phenyl)-5-phenyl-1H-pyrrol-2-yl)imino)-5-phenyl;2H-pyrrol-3-yl)phenoxy)acetate (8)

In a flask under argon are dissolved 0.183 g of azadipyrromethene 7 (0.32 mmol, 1 eq.) and 0.548 ml of DIPEA (3.22 mmol, 10 eq.) in freshly distilled dichloromethane. After stirring for a few min at room temperature, 0.613 ml of distilled BF₃.Et₂O (4.84 mmol, 15 eq.) are added and the solution is refluxed for two hours. The solution is then cooled to room temperature and the organic phase is washed with brine three times. The aqueous phase is then re-extracted three times with dichloromethane and the combined organic phases are dried over MgSO₄, filtered and concentrated in vacuo. The residue is purified by chromatography on silica with a gradient of PE/DCM. Azabodipy 8 is obtained as an iridescent dark blue powder (0.196 g, 0.318 mmol, 99%).

¹H NMR (400 MHz, Acetone-d₆) δ8.23 (dd, J=8.8, 6.2 Hz, 4H), 8.18-8.09 (m, 4H), 7.58-7.46 (m, 6H), 7.33 (d, J=1.4 Hz, 2H), 7.14 (dd, J=9.0, 7.2 Hz, 4H), 4.88 (s, 2H);), 3.92 (s, 3H), 3.79 (s, 3H).

¹³C NMR (101 MHz, acetone) δ169.66, 162.39, 160.53, 132.72, 131.94, 131.87, 131.70, 131.62, 130.55, 130.51, 129.36, 125.87, 118.92, 115.83, 115.26, 65.70, 55.88, 52.29.

¹⁹F NMR (235 MHz, Acetone-d₆) δ−130.38 (dd, J=62.8-31.4 Hz).

HRMS (ESI): m/z calcd for [C₃₆H₂₉BF₂N₃O₄]: 616.221996, measured 616.221217 (−1.3 ppm)

Mp: 179° C.

IR (cm⁻¹): 3288, 2918, 2584, 1758, 1728, 1601, 1504, 1487, 1454, 1388, 1277, 1252, 1228, 1175, 1129, 1100, 1068, 1024, 999, 970, 929, 904, 868, 836, 818, 767, 742, 690, 641, 615.

### Synthesis of (Z)-2-(4-(2((1-(difluoroboryl)-3-(4-methoxyphenyl)-5-phenyl-1H-pyrrol-2-yl)imino)-5-phenyl-2H-pyrrol-3-yl)phenoxy)acetic acid (9)

0.308 g of azabodipy 8 (0.5 mmol, 1 eq.) in a THF/water/H₃PO₄ mixture (50 ml: 25 ml: 10 ml) is dissolved in a flask. The solution is stirred under reflux for 20 hours until no trace of the ester is visible by TLC. After cooling, the solution is extracted with dichloromethane. The organic phase is washed with brine and then the aqueous phases are re-extracted with dichloromethane until no blue color is observed in the aqueous phase. The organic phase is dried over MgSO₄, filtered and concentrated under reduced pressure. The residue may optionally be purified by chromatography with DCM/MeOH if the ester is still present in the crude. Azabodipy 9 is obtained as a dark blue solid (0.294 g, 0.49 mmol, 98%).

¹H NMR (250 MHz, Acetone-d₆) δ8.27-8.18 (m, 4H), 8.18-8.09 (m, 4H), 7.57-7.48 (m, 6H), 7.32 (d, J=1.8 Hz, 2H), 7.19-7.08 (m, 4H), 4.86 (s, 2H), 3.91 (s, 3H).

HRMS (ESI): m/z calcd for [C₃₅H₂₇BF₂N₃O₄]: 602.206329, measured 602.205753 (1.0 ppm)

### Synthesis of tert-butyl N-[2-[[2-[4-[(2Z)-2-[1-difluoroboranyl-3-(4-methoxyphenyl)-5-phenyl-pyrrol-2-yl]imino-5-phenyl-pyrrol-3-yl]phenoxy]acetyl]amino]ethyl]carbamate (10)

In a flask under argon are dissolved 0.080 g of 9 (0.13 mmol, 1 eq.), 0.070 ml of DIPEA (0.40 mmol, 3 eq.) and 0.076 g of HBTU (0.20 mmol, 1.5 eq.) in 5 mL of dichloromethane and 1 mL of distilled acetonitrile. The reaction is stirred for 15 min and then 0.032 g of Boc-ethylene diamine (0.20 mmol, 1.5 eq.) dissolved in 2 mL of anhydrous dichloromethane are added. The reaction is stirred 1:30. Then, the solution is extracted with dichloromethane and washed successively with 1M KHSO₄, NaHCO₃ sat. and NaCl sat. The organic phases are combined and dried over MgSO₄, filtered and concentrated in vacuo. The residue is then purified by chromatography on silica with a PE/EA mixture giving 0.069 g of a deep blue solid with metallic reflections (0.093 mmol, 71%).

¹H NMR (250 MHz, Chloroform-d) δ8.10-7.98 (m, 8H), 7.51-7.44 (m, 6H), 7.02 (dd, J=9.0, 8), 1 Hz, 4H), 6.94 (d, J=1.1 Hz, 2H), 4.88 (s, 1H), 4.58 (s, 2H), 3.92 (s, 3H), 3.50 (q, J=5.6 Hz, 2H), 3.35 (dd, J=12.1, 6.0 Hz, 2H), 2.80 (s, 3H), 1.43 (s), 9H).

¹³C NMR (101 MHz, CDCl₃) δ131.13, 131.08, 130.96, 130.79, 129.66, 128.69, 125.36, 117.98, 117.94, 115.03, 114.42, 67.41, 55.65, 38.76, 28.49.

HRMS: [M+H]⁺ C₂₄H₄₁BF₂N₅O₅ m/z calculated 744.317054, measured 744316900 (0.2 ppm).

### 1.2. Preparation of 2-[4-[(2Z)-2-[5-[4-[4-(tert-butoxycarbonylamino) butoxy]phenyl]-1-difluoroboranyl-3-phenyl-pyrrol-2-yl]imino-5-phenyl-pyrrol-3-yl]phenoxy]methylacetate (19)

This antenna (19) is obtained according to the reaction scheme below:

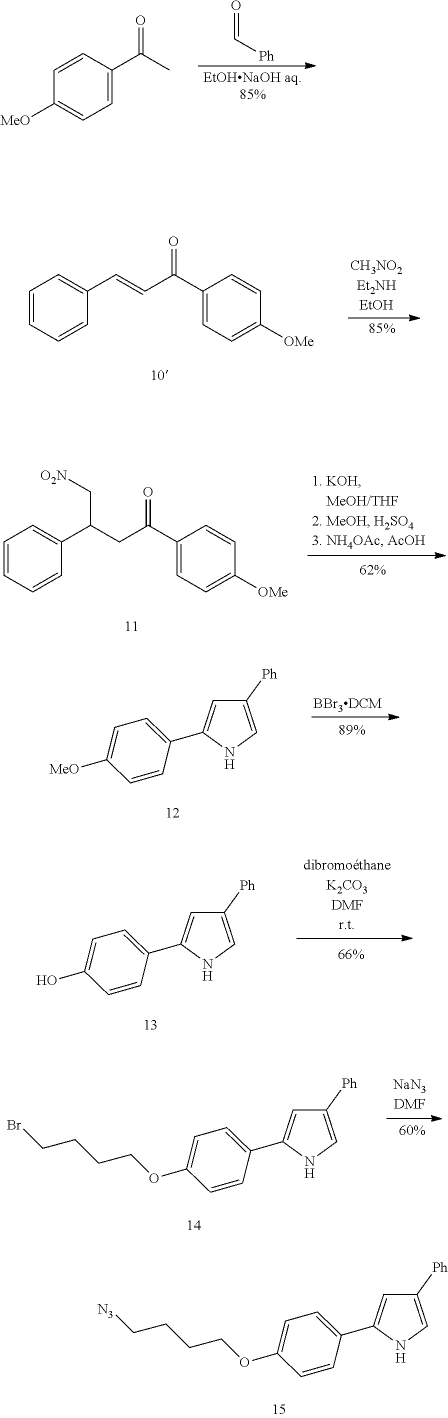

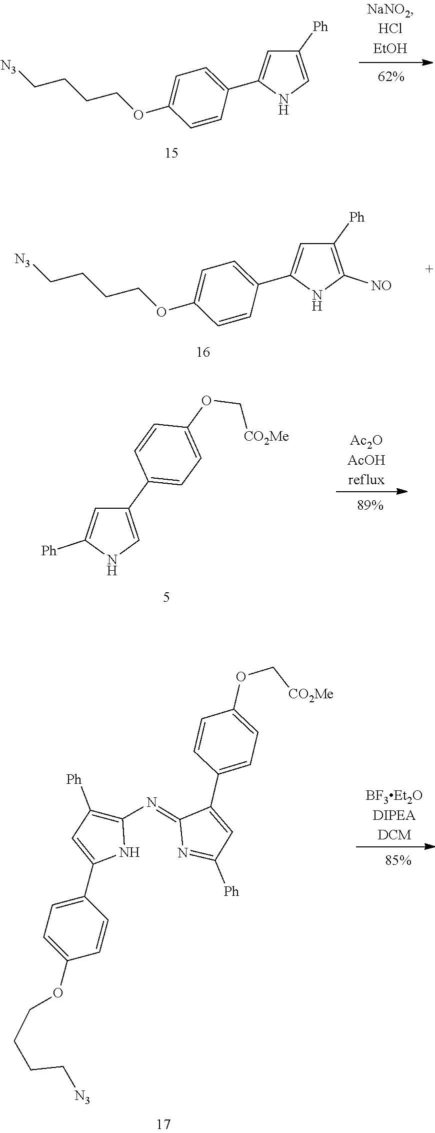

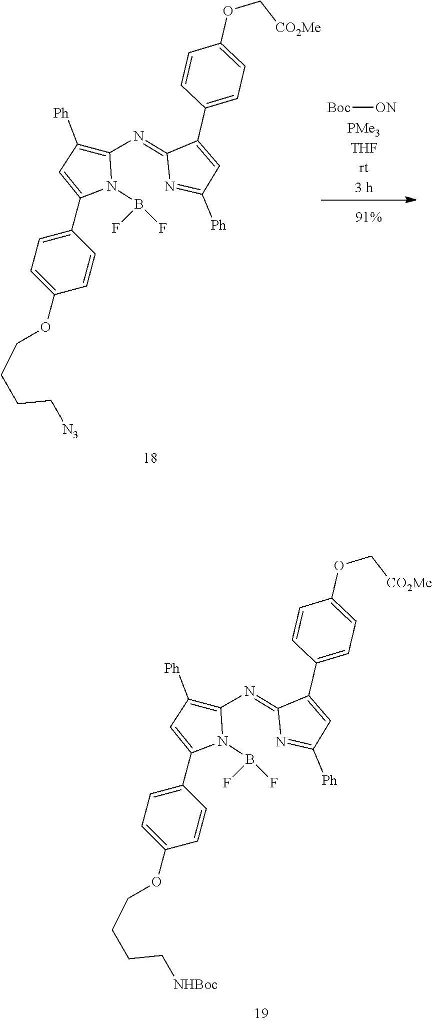

### Synthesis of (E)-1-(4-methoxyphenyl)-3-phenyl-prop-2-en-1-one (10′)

15 g of paramethoxyacetophenone (0.1 mol, 1 eq.), 10.1 ml of benzaldehyde (0.1 mol, 1 eq.) and 400 mg of NaOH (10 mmol, 0.1 eq.) in methanol. The solution is stirred at reflux overnight. Cold water is then added to the mixture and the precipitate formed is filtered and washed with water. The chalcone 10′ is recrystallized from methanol as white crystals in 85% yield (20.254 g, 85 mmol).

¹H NMR (CDCl₃, 250 MHz): 8.08-8.01 (tt, 2H), 7.80 (d, J=15.7 Hz, 1H), 7.67-7.61 (m, 2H), 7.54 (d, J=15.7 Hz, 1H), 7.44-7.37 (m, 3H), 7.01-6.94 (tt, 2H), 3.87 (s, 3H).).

### Synthesis of 1((4-methoxyphenyl)-4-nitro-3-phenyl-butan-1-one (11)

This compound is synthesized by applying the procedure described for the preparation of compound 2.

Mass obtained: 3.205 g; 10.71 mmol

Yield 85%.

¹H NMR (CDCl₃, 400 MHz): 7.88-7.86 (d, 2H), 7.31-7.21 (m, 5H), 6.90-6.88 (d, 2H), 4.80 (ddd, J=12.6 Hz, 6.4 Hz, 1.3 Hz, 1H), 4.65 (ddd, J=12.6 Hz, 8.3 Hz, 1.3 Hz), 4.18. (p, J=7.1Hz, 1H), 3.82 (s, 3H), 3.42-3.29 (m, 2H).

¹³C NMR (CDCl₃, 101 MHz): 195.39, 163.88, 139.39, 130.39, 129.52, 129.06, 127.83, 127.51, 113.93, 79.68, 55.56, 41.22, 39.49.

### Synthesis of 2-(4-methoxyphenyl)-4-phenyl-1H-pyrrole (12)

This compound is synthesized by applying the procedure described for the preparation of compound 3.

Mass obtained: 1.917 g; 7.69 mmol

Yield: 62%
¹H NMR (CDCl₃, 250 MHz): 8.37 (s, 1H), 7.57 (dd, J=8.4 Hz, 1.3 Hz, 2H), 7.48-7.42 (m, 2H), 7.36 (t, J=7.5 Hz, 3H), 7.23-7.16 (m, 1H), 7.11 (dd, J=2.6 Hz, 1.7 Hz, 1H)), 6.98-6.91 (m, 2H), 6.71 (dd, J=2.7 Hz, 1.7 Hz, 1H), 3.84 (s, 3H).

¹³C NMR (CDCl₃, 63 MHz): 158.63, 135.79, 133.27, 128.77, 126.64, 125.79, 125.73, 125.46, 125.31, 115.00, 114.55, 103.14, 55.51.

### Synthesis of 4-(4-phenyl-1 H-pyrrol-2-yl)phenol (13)

This compound is synthesized by applying the procedure described for the preparation of compound 4.

Mass obtained: 180 mg, 0.77 mmol

Yield: 89%

Mp: >300° C.

HRMS: [M+H]⁺: 236.1070.

IR (cm⁻¹): u=3215, 1495, 1249, 1171, 1101, 832, 763, 696

1H NMR (Acetone-d6, 250 MHz): 10.41 (s, 1H), 8.29 (s, 1H), 7.62-7.58 (m, 2H), 7.55-7.51 (m, 2H), 7.33-7.27 (m, 2H), 7.22 (dd, J=2.8 Hz, 1.7 Hz, 1H), 7.15-7.08 (m, 1H), 6.89-6.85 (m, 2H), 6.77 (dd, J=2.8 Hz, 1.8 Hz, 1H).

### Synthesis of 2-[4-(4-bromobutoxy)phenyl]-4-phenyl-1H-pyrrole (14)

0.450 g of 13 (1.91 mmol, 1 eq.), 0.794 g of K₂CO₃ (5.74 mmol, 3 eq.) and 0.69 ml of 1,4-dibromobutane (5 g) are dissolved in a flask at room temperature. 74 mmol, 3 eq.) in DMF. The reaction is stirred vigorously at room temperature overnight. The next day, water is added to the reaction and the mixture is extracted with diethyl ether, the organic phase is washed 3 times with sat. NaCl and the aqueous phases are re-extracted with diethyl ether. The organic phases are combined, dried over MgSO₄, filtered and concentrated in vacuo. The residue is dissolved in a minimum of dichloromethane, and the product is precipitated by addition of petroleum ether, filtered through Buchner and washed with petroleum ether giving 0.466 g of 14 (1.26 mmol, 66%) form of a white powder.

Mp: 160-162° C.

HRMS: [M(⁷⁹Br)+H]⁺: 371.1813, [M(⁸¹Br)+H]⁺: 373,0814, [M+H]⁺: 372.0781.

IR (cm⁻¹): u=3395, 1497, 1247, 830, 802, 751, 692.

¹H NMR (DMSO-d6, 250 MHz): 11.28 (s, 1H,), 7.61-7.57 (m, 4H), 7.34-7.31 (d, 2H), 7.28-7.25 (dd, 1H), 7.14-7.07 (tt, 1H), 6.95 (d, J=2.8 Hz, 2H), 6.80 (dd, J=2.7 Hz, 1.7 Hz, 1H), 4.02 (t, J=6.1 Hz, 2H), 3.62 (t, J=6.5 Hz, 2H), 2.01-1.94 (m, 2H), 1.88-1.81 (m, 2H).

¹³C NMR (DMSO-d₆, 63 MHz): 156.82, 135.86, 132.27, 128.48, 125.59, 124.88, 124.76, 124.49, 124.33, 115.71, 114.71, 101.94, 66.59, 34.86, 29.11, 27.42.

### Synthesis of 2-[4-(4-azidobutoxy) phenyl]-4-phenyl-1H-pyrrole (15)

0.450 g of 14 (1.22 mmol, 1 eq.) and 0.395 g of sodium azide (6.08 mmol, 5 eq.) in DMF are dissolved in a flask. The reaction is stirred at room temperature overnight. The mixture is dissolved in dichloromethane and washed several times with sat. NaCl. The organic phase is dried over MgSO₄, filtered and concentrated in vacuo. After dilution with a minimum of dichloromethane, the product is precipitated by the addition of petroleum ether. The solid is filtered on Buchner and 243 mg (0.73 mmol, 60%) is obtained as a white powder.

Mp: 177-180° C.

HRMS: [M+H]⁺: 333.1707.

IR (cm⁻¹): u=3394, 2948, 2875, 2080, 1496, 1246, 831, 752, 692.

¹H NMR (CDCl₃, 250 MHz): 8.38 (s, 1H), 7.59-7.55 (m, 2H), 7.45-7.39 (m, 2H), 7.37-7.33 (m, 2H), 7.23-7.17 (m, 1H), 7.10-7.08 (m, 1H), 6.94-6.88 (m, 2H), 6.72 (dd, J=2.6 Hz, 1.5 Hz, 1H), 4.01 (t, J=5.8 Hz, 2H), 3.38 (t, J=6.3 Hz, 2H), 1, 89-1.80 (m, 4H)

¹³C NMR (CDCl₃, 63 MHz): 157.86, 135.76, 133.21, 128.77, 126.57, 125.89, 125.77, 125.41, 125.27, 116.09, 115.09, 103.09, 67.41, 51.34, 26.66, 25.90.

### Synthesis of 5-[4-(4-azidobutoxy)phenyl]-2-nitroso-3-phenyl-1H-pyrrole (16)

This compound (ocher-golden powder) is synthesized by applying the procedure described for the preparation of compound 6.

Mass obtained: 67 mg, 0.186 mmol

Yield: 62%

Melting point: 125-126° C.

HRMS: [M+H]⁺: 362.11612.

IR (cm⁻¹): u=3280, 2918, 2850, 2092, 1603, 1360, 1258, 1164, 1038, 828, 768, 695, 668.

¹H NMR (CDCl₃, 250 MHz): 8.14-8.11 (m, 2H), 7.84 (d, J=8.4 Hz, 2H), 7.46 (dd, J=5.2 Hz), 1.9 Hz, 3H), 7.15 (s, 1H), 6.99 (d, J=8.2 Hz, 2H), 4.06 (t, J=5.8 Hz, 2H), 3.39 (t, J=6.4 Hz, 2H), 1.93-1.78 (m, 4H).

¹³C NMR (CDCl₃, 63 MHz): 163.72, 162.05, 142.84, 131.91, 129.71, 129.57, 129.29, 128.93, 122.19, 115.47, 115.03, 67.67, 51.27, 26.53, 25.82.

### Synthesis of methyl 2-[4-[(2Z)-2-[[5-[4-(4-azidobutoxy)phenyl]-3-phenyl-1H-pyrrol-2-yl]imino]-5-phenylpyrrol3-yl]phenoxy]acetate (17)

This compound (intense blue powder) is synthesized by applying the procedure described for the preparation of compound 7.

Mass obtained: 0.080 g, 0.123 mmol

Yield: 89%.

Mp: 150-152° C.

HRMS: [M+H]⁺: 651.2717.

IR (cm⁻¹): u=2094, 1759, 1600, 1496, 1241, 1166, 903, 806, 764, 694, 675.

¹H NMR (CDCl₃, 400 MHz): 8.00 (d, J=7.8 Hz, 4H), 7.92 (d, J=8.3 Hz, 2H), 7.80 (d, J=7), 6 Hz, 2H), 7.48 (t, J=7.5 Hz, 2H), 7.39 (dd, J=12.8 Hz, 6.9 Hz, 4H), 7.17 (s, 1H), 6.99 (d, 3H), 6.93 (d, J=8.4 Hz, 2H), 4.69 (s, 2H), 4.06 (t, J=6.1 Hz, 2H), 3.85 (s, 3H), 3.40 (t, J=6.6 Hz, 2H), 1.91 (dt, J=11.4 Hz, 6.1 Hz, 2H), 1.82 (p, J=6.8 Hz, 2H).

¹³C NMR (CDCl₃, 101 MHz): 169.49, 161.27, 160.76, 157.61, 153.83, 148.27, 145.30, 145.27, 138.66, 133.76, 131.94, 130.36, 129.28, 129.22, 129.00, 128.32, 128.22, 128.15, 125.91, 125.50, 117.43, 115.16, 114.52, 111.16, 67.56, 65.53, 52.45, 51.32, 26.61, 25.87.

### Synthesis of 2-[4-[(2Z)-2-[5-[4-(4-azidobutoxy)phenyl]-1-difluoroboranyl-3-phenylpyrrol-2-yl]imino-5-phenylpyrrol3-yl]phenoxy]methylacetate(18)

This compound (intense green solid of metallic appearance) is synthesized by applying the procedure described for the preparation of compound 8.

Mass obtained: 0.065 g, 0.093 mmol

Yield: 85%

¹H NMR (400 MHz, Acetone-d₆): δ8.27-8.19 (m, 6H), 8.16-8.11 (m, 2H), 7.60 -7.46 (m, 7H), 7.29 (s, 1H), 7.14-7.08 (m, 4H), 4.87 (s, 2H), 4.24-4.17 (m, 2H), 3.78 (d), J=2.1 Hz, 3H), 3.47 (t, J=6.5 Hz, 2H), 1.98-1.89 (m, 2H), 1.83 (q, J=7.4 Hz, 2H).

¹³C NMR (101 MHz, Acetone): δ133.16, 131.74, 131.33, 130.48, 130.43, 130.29, 129.60, 129.34, 118.67, 115.76, 115.65, 68.49, 65.69, 52.28, 51.76, 27.13, 26.32.

HRMS: [M+H]⁺C₃₉H₃₄BF₂N₆O₄ m/z calculated 699.270390; measured 699.270354 (0.1 ppm)

### Synthesis of 2-[4-[(2Z)-2-[5-[4-[4-(tert-butoxycarbonylamino) butoxy]phenyl]-1-difluoroboranyl-3-phenyl-pyrrol-2-yl]imino-5-phenyl-pyrrol-3-yl]phenoxy]methylacetate (19)

0.061 g of 18 (0.087 mmol, 1 eq.) and 0.024 g of Boc-ON (0.096 g, 1.1 eq.) in anhydrous THF are dissolved under argon in a temperature-controlled flask. To this solution are added 0.096 ml of a 1M solution of trimethylphosphine in toluene (0.096 mmol, 1.1 eq.). The solution is stirred overnight at room temperature. The next day, the solution is checked by TLC and if the starting material is still visible, 1 eq. is added. additional PMe₃ and Boc-ON to complete the reaction in one hour. The solution is then washed with sat. NaCl and extracted with dichloromethane. The organic phase is dried over MgSO₄, filtered and concentrated in vacuo. The residue is purified by chromatography on silica (PE/EA) and 0.061 g of 19 (0.079 mmol, 91%) are obtained in the form of an iridescent dark green solid.

¹H NMR (400 MHz, Acetone-d₆) δ8.26-8.17 (m, 6H), 8.15-8.09 (m, 2H), 7.52 (td, J=9.6, 8), 9, 4.8 Hz, 7H), 7.25 (s, 1H), 7.11-7.05 (m, 4H), 6.00 (s, 1H), 4.85 (s, 2H) , 4.16 (dt, J=6.6, 3.4 Hz, 2H), 3.78 (s, 3H), 3.17 (td, J=7.6, 3.8 Hz, 2H), 1.84 (q, J=7.1 Hz, 2H), 1.69 (p, J=7.4 Hz, 2H), 1.41 (s, 9H).

¹³C NMR (101 MHz, Acetone) δ169.64, 163.11, 160.51, 160.29, 158.35, 156.72, 146.61, 145.53, 144.69, 143.18, 13312, 133.07, 133.04, 131.70, 131.27, 130.44, 130.41, 130.37, 130.26, 129.55, 129.31, 126.86, 124.28 , 120.79, 118.57, 115.71, 115.62, 78.39, 68.77, 65.66, 52.28, 41.83, 40.75, 28.68, 27.43, 27.21, 24.44.

HRMS: [M+H]⁺C₄₂H₄₁BF₂N₅O₅ calculated 744.317054, measured 744.316900 (0.2 ppm).

### 2. Preparation of Perylenediimide Antennas

### Synthesis of the Clickable Perylenebisimide Derivative

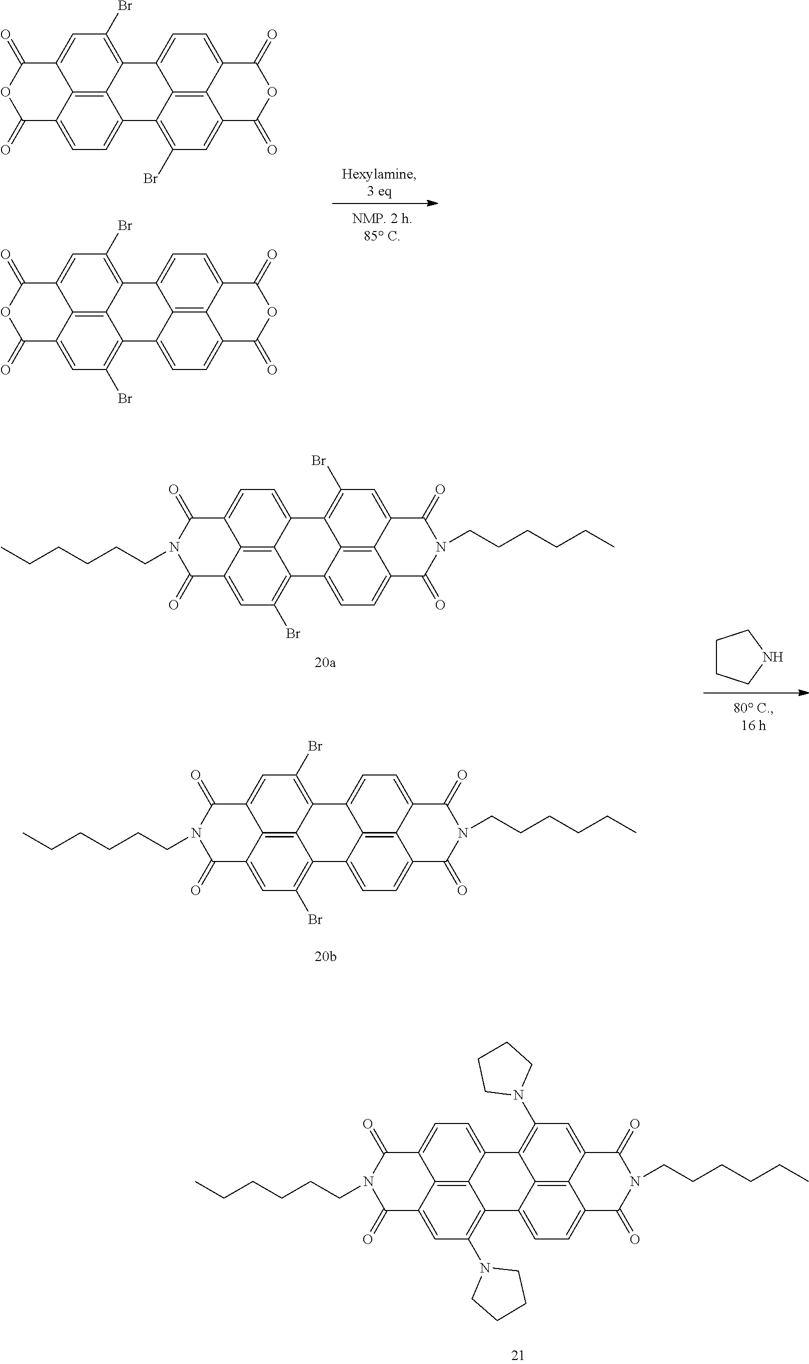

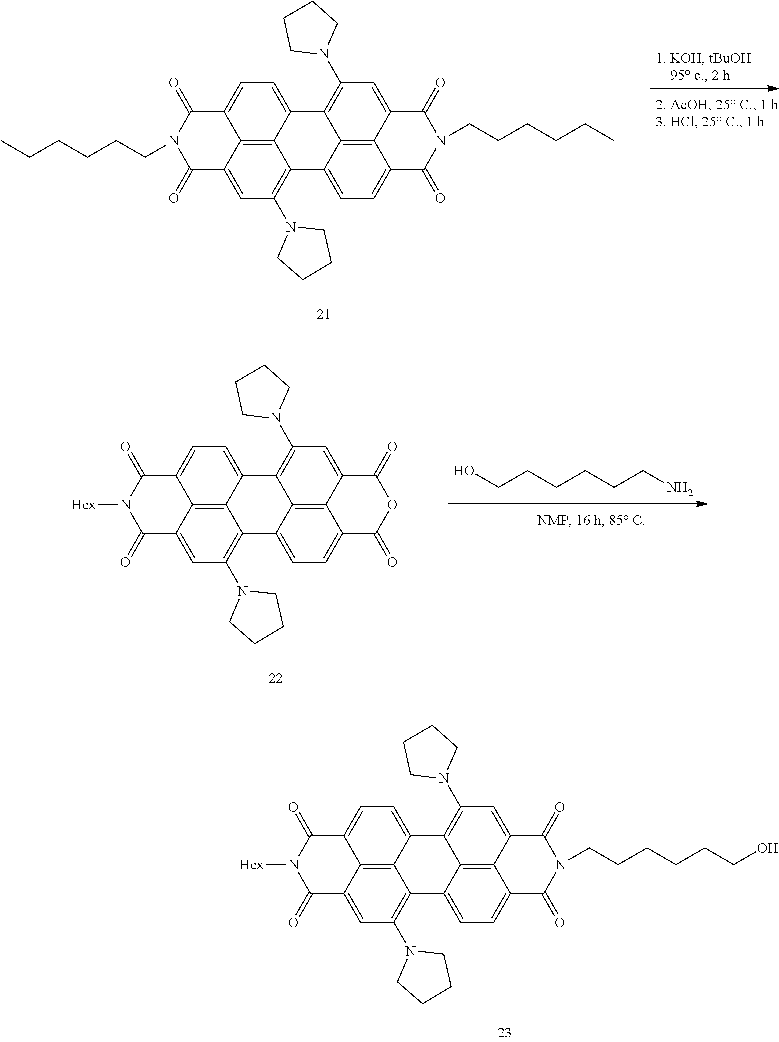

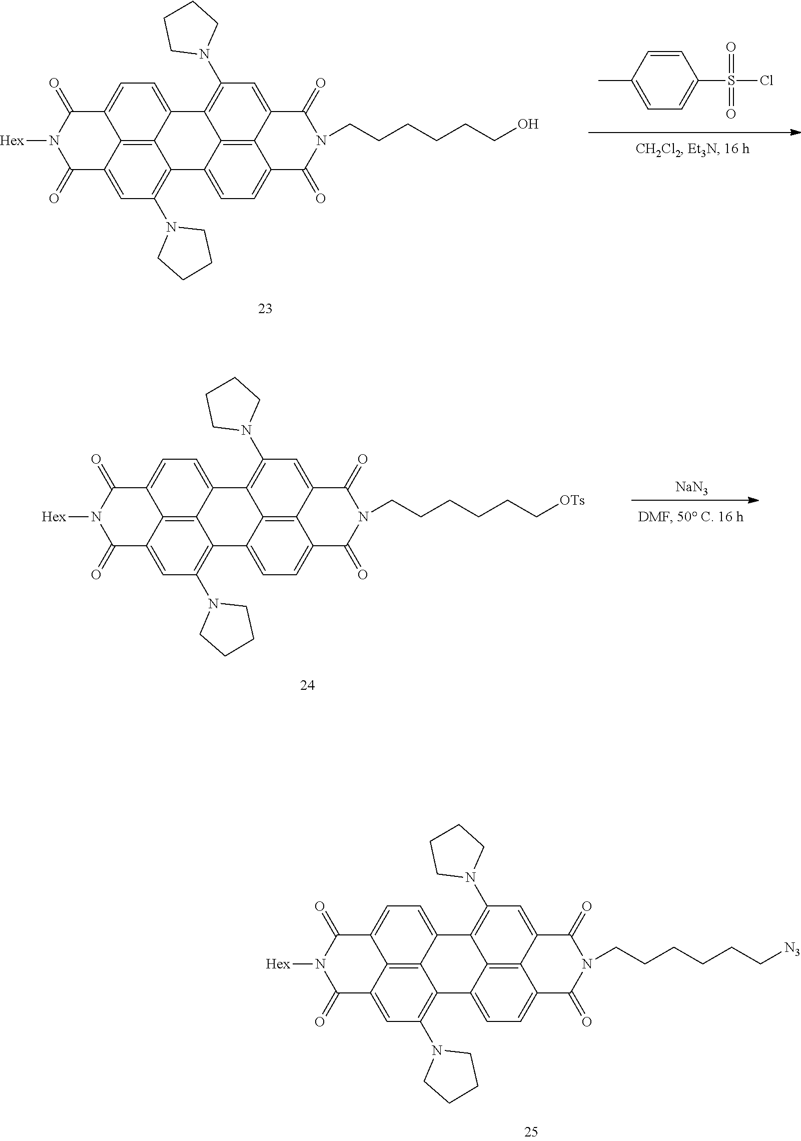

Compounds 20a-20b

The mixture 1,7- and 1,6-dibromoperylene-3,4,9,10-tetracarboxylic dianhydride (1.03 g, 1.8 mmol, 1.0 eq.) is solubilized under argon in 20 ml of NMP. To this solution are added hexylamine (0.546 g, 5.4 mmol, 700 μL, 7.3 eq.) and acetic acid (0.432 g, 7.2 mmol, 412 μL, 4.0 eq.). The reaction mixture is stirred at 85° C. under an inert atmosphere for 2 h. After returning to ambient temperature, the reaction mixture is poured into ethanol. The red precipitate is filtered in vacuo and washed several times with ethanol. The desired mixture P1a and P1b is obtained in the form of a red powder which is used directly in the next reaction.

Compound 21

The mixture obtained above (20a, 20b) is solubilized in pyrrolidine. The solution is stirred under an inert atmosphere at 85° C. for 16 h. At the end of the reaction, the solution is cooled and a solution of 1M HCl is added until pH=2. The aqueous phase is extracted three times with dichloromethane. The organic phases are combined, dried over Na₂SO₄, filtered and evaporated under reduced pressure. The crude reaction product is purified on a silica gel chromatography column (Eluent: Hexane/ethyl acetate (7/3)) to give the 1.7 (P2) isomer in the form of a green powder.

¹H NMR (600MHz, CDCl₃): 8.37 (s, 2H), 8.31 (d, J=8.0 Hz, 2H), 7.53 (d, J=8.0 Hz, 2H), 4H NMR (CDCl3):?, 21 (dd, J=8.6, 6.6 Hz, 2H), 3.68 (brs, 4H), 2.75 (brs, 4H), 2.04 (brs, 4H), 1.94 (b.p. brs, 4H), 1.75 (m, 4H), 1.46 (m, 4H), 1.37-1.34 (m, 8H), 0.90 (t, J=7.0 Hz, 6H).

¹³C NMR (151 MHz, CDCl₃): 164.05, 146.39, 134.10, 129.82, 126.55, 123.71, 122.03, 121.66, 120.66, 119.00, 117, 98, 52.14, 40.54, 31.65, 28.16, 26.87, 25.80, 22.61, 14.11.

Compound 22

Compound 21 (180 mg, 2.58.10⁻⁴ mol, 1.0 eq.) is solubilized in tBuOH (25 mL). To this solution is added the milled KOH (73 mg, 1.29.10⁻³, 5.0 eq.). The reaction mixture is stirred under an inert atmosphere at 95° C. for 3 h. The progress of the reaction is monitored by TLC. At the end of the reaction, the reaction mixture is cooled and 10 mL of acetic acid are added. The reaction mixture is stirred for a further 1 h, then a solution of 1M HCl (10mL) is added. The reaction mixture is stirred for an additional hour. Dichloromethane is then added and the organic phase is washed three times with water. The organic phase is then dried over Na₂SO₄, filtered and evaporated under reduced pressure. The crude reaction product is purified on a chromatographic column (eluent: hexane/ethyl acetate (6/4)) to give compound P3 in the form of a green powder.

¹H NMR (600MHz, CDCl₃): 8.49 (s, 1H), 8.43 (s, 1H), 8.42 (d, J=8.0 Hz, 2H), 8.40 (d, J=8.06 Hz, 2H), 7.69 (d, J=8.01 Hz, 2H), 7.55 (d, J=8.03 Hz, 2H), 4.22 (m, 2H), 3.74 (brs, 4H), 2.83 (brs, 4H), 2.11 (brs, 4H), 2.00 (brs, 4H), 1.76 (m, 2H), 1.45 (m, 2H), 1.36-1.30 (m, 8H), 0.90 (t, J=6.98 Hz, 3H)

Compound 23

Compound 22 (180 mg, 2.58.10⁻⁴ mol, 1.0 eq.) is solubilized in 10 mL of NMP and 6-amino-1-hexanol (87 mg, 1.4.10⁻⁴ mol, 1.0 eq.) is added. The reaction mixture is stirred under an inert atmosphere at 85° C. for 16 h. At the end of the reaction, the reaction mixture is cooled and dichloromethane is then added. The organic phase is washed three times with water. The organic phase is then dried over Na₂SO₄, filtered and evaporated under reduced pressure. The crude reaction product is purified on a chromatographic column (Eluent: Hexane/ethyl acetate (7/3)) to give the compound P4 in the form of a green powder.

¹H NMR (600 MHz, CDCl₃): 8.51 (s, 1H), 8.50 (s, 1H), 8.45 (dd, J=8.1, 1.8Hz, 2H), 7.74 (dd, J=8.0, 6.9 Hz, 2H), 4.23 (m, 4H) 3.77 (brs, 4H), 2.85 (brs, 4H), 2.38 (t, J=8.1 Hz, 2H), 2.10 (brs, 4H), 2.00 (brs, 4H), 1.79-17.6 (m, 4H), 1.57 (m, 4H), 1.49-1.44 (m, 4H), 1.38-1.35 (m, 4H), 0.9 (t, J=7.0 Hz, 3H).

Compound 24

Compound 23 (135 mg, 1.9.10⁻⁴ mol, 1.0 eq.) is solubilized in dry dichloromethane (20 mL). To this solution are added triethylamine (256 μL, 1.9 mmol, 10.0 eq.) and 4-toluenesulfonyl chloride (366 mg, 1.9 mmol, 10.0 eq.). The reaction mixture is stirred at ambient temperature under an inert atmosphere for 16 h. At the end of the reaction, water is added and the organic phase is washed three times with water. The organic phase is then dried over Na₂SO₄, filtered and evaporated under reduced pressure. The crude reaction product is purified on a chromatographic column (Eluent: Hexane/ethyl acetate (8/2)) to give the compound P5 in the form of a green powder.

¹H NMR (600 MHz, CDCl₃): 8.50 (d, J=6.5 Hz, 2H), 8.44 (dd, J=8.09, 6.9 Hz, 2H), 7.79 (m, 2H), 7.73 (t, J=7.8 Hz, 2H), 7.35 (d, J=8.0 Hz, 2H), 4.22 (dt, J=22.9, 7.6); Hz, 4H), 4.03 (t, J=6.5 Hz, 2H), 3.77 (brs, 4H), 2.85 (brs, 4H), 2.44 (s, 3H), 2.10 (brs, 4H), 1.99 (brs, 4H), 1.77-1.66 (m, 6H), 1.45 (t, J=7.6 Hz, 2H), 1.40-1.33 (m, 8H), 0.90 (t, J=7.0 Hz, 3H).

¹³C NMR (151 MHz, CDCl₃): 164.18, 146.58, 144.76, 134.23, 133.34, 129.99, 129.95, 128.01, 126.73, 126.69, 12.91, 123.84, 122.19, 121.86, 120.82, 119.20, 119.01, 118.21, 118.05, 70.71, 52.30, 40.67, 40.30, 31.77, 28.89, 28.29, 28.05, 26.99, 26.61, 25.93, 25.29, 22.74, 21.77, 14.24.

Compound 25

Compound 24 (68 mg, 78.10⁻³ mol, 1.0 eq.) is solubilized in 20 mL of DMF. To this solution is added sodium azide (51 mg, 7.8.10⁻³ mol, 10.0 eq.). The reaction mixture is stirred under an inert atmosphere at 50° C. for 12 h. At the end of the reaction, the reaction mixture is cooled and water is added. The aqueous phase is extracted three times with dichloromethane. The organic phases are pooled, dried over Na₂SO₄, filtered and evaporated under reduced pressure to yield the desired product P6 without further purification.

¹H NMR (600MHz, CDCl₃): 8.38 (d, J=2.3Hz, 2H), 8.33 (dd, J=8.0, 6.4Hz, 2H), 7.55 (dd, J=12.3, 8.0 Hz, 2H), 4.22 (q, J=7.4 Hz, 4H), 3.69 (brs, 4H), 3.28 (t, J=7.0 Hz, 2H), 2.76 (brs, 4H), 2.05 (brs, 4H), 1.95 (brs, 4H), 1.84-1.72 (m, 6H), 1.65 (t, J=7.0 Hz, 3H), 1.48 (m, 7H), 1.36 (m, 5H), 0.90 (t, J=7.0 Hz, 3H).

¹³C NMR (151 MHz, CDCl₃): 164.20, 146.56, 134.31, 134.20, 129.96, 126.73, 123.89, 122.18, 122.16, 121.85, 121.29, 120.80, 119.19, 119.02, 118.19, 118.05, 52.29, 51.54, 40.68, 40.39, 31.77, 29.83, 29.65, 28.88, 28.29, 28.13, 27.00, 26.81, 26.63, 25.93, 22.74, 14.24.

### 3. Preparation of Water-Solubilising Groups

### Synthesis of compound 27

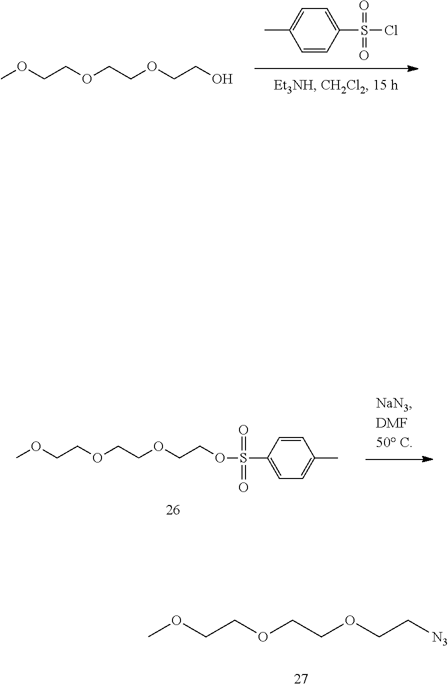

Compound 26

Triethylene glycol mono methyl ether (2.0 g, 1.22.10⁻² mol, 1.0 eq.) is solubilized in 120 mL of dry CH₂Cl₂. To this solution are added under argon 4-toluenesulfonyl chloride (4.64 g, 2.43.10⁻² mol, 2.0 eq.) and triethylamine (2.68 g, 2.43.10⁻² mol, 2.0 g). eq.). The reaction mixture is stirred under an inert atmosphere at ambient temperature for 16 h. At the end of the reaction, water is added to the reaction mixture and the organic phase is washed three times with water, dried over Na₂SO₄, filtered and evaporated under vacuum. The reaction crude is purified by chromatography column on silica gel (Eluent: CH₂Cl₂/MeOH (0.5%)) to give the desired product in the form of a colorless oil.

¹H NMR (600MHz, CDCl₃): 7.79 (d, J=8.3 Hz, 2H), 7.33 (d, J=8.0 Hz, 2H), 4.20-4.10 (m, 2H), 3.72-3.64 (m, 2H), 3.63-3.56 (m, 6H), 3.55-3.49 (m, 2H), 3.36 (s, 3H); , 2.44 (s, 3H).

¹³C NMR (151 MHz, CDCl₃): 144.90, 133.14, 129.93, 121.10, 72.03, 70.87, 70.69, 70.67, 69.35, 68.80, 59.15, 21.76.

Compound 27

Compound 26 (2.87 g, 9.02 mmol, 1.0 eq.) is solubilized in 90 mL of dry DMF. To this solution is added under an inert atmosphere, NaN₃ (4.70 g, 72.0 mmol, 8 eq.). The reaction mixture is stirred at 50° C. for 16 h. After cooling to room temperature, water is added to the reaction mixture and the aqueous phase is extracted with CH₂Cl₂. The organic phase is washed with water, dried over Na₂SO₄ filtered and evaporated under reduced pressure. The desired product is obtained in the form of an oil without further purification.

¹H NMR (600 MHz, CDCl₃): 3.69-3.65 (m, 8H), 3.57-3.55 (m, 2H), 3.40-3.39 (m, 5H).

¹³C NMR (151 MHz, CDCl₃): 72.09, 70.86, 70.82, 70.77, 70.19, 59.19, 50.84.

### 4. Preparation of Porphyrin Type Antennas

Tetraphenylporphyrin (TPPH, compound 28) was synthesized following the Alder-Rothemund method from a mixture of benzaldehyde and pyrrole in propionic acid, heated at 130° C. for two hours. The crystalline product was isolated by filtration in 6% yield. The low yield is in agreement with the literature and the synthetic route used.

The next step is an electrophilic aromatic substitution, also called aromatic nitration. The latter is controllable regioselectively in the para position of the phenyl, by varying the amount of sodium nitrite and the reaction time in the TFA. In fact, after concentrating TPPH in TFA, the latter was treated with 1.8 equivalents of sodium nitrite for exactly 3 min. The reaction mixture was re-engaged without intermediate purification in order to reduce the nitro group to an amino group in the presence of excess of tin chloride and hydrochloric acid. The mono-amino unsymmetric porphyrin (compound 29) was isolated by column chromatography (silica, DCM/Hexane, 9:1).

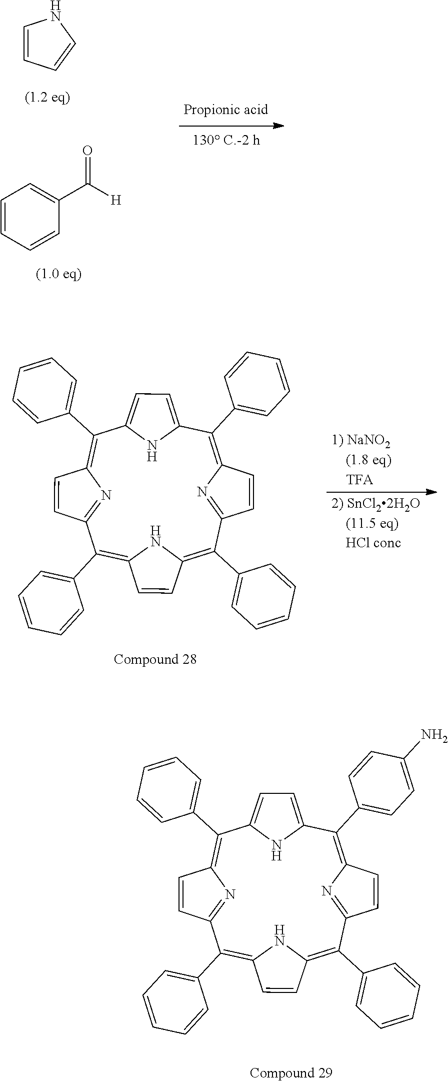 [SMILES: c1cc[nH]c1 ; O=Cc1ccccc1 ; C1=Cc2nc1c(-c1ccccc1)c1ccc([nH]1)c(-c1ccccc1)c1nc(c(-c3ccccc3)c3ccc([nH]3)c2-c2ccccc2)C=C1 ; Nc1ccc(-c2c3nc(c(-c4ccccc4)c4ccc([nH]4)c(-c4ccccc4)c4nc(c(-c5ccccc5)c5ccc2[nH]5)C=C4)C=C3)cc1]

5,10,15,20-Tetraphenylporphyrin (TPPH, Compound 28)

Benzaldehyde (5.0 mL, 49.1 mmol, 1.2 eq.) and pyrrole (4.0 mL, 57.6 mmol, 1.0 eq.) were added in 250 mL of propionic acid. The solution was stirred at 130° C. for 2 h. The reaction mixture was then cooled to room temperature and filtered. The purple solid was washed with MeOH and hot water to give the desired product as a purple powder (1.70 g, 2.76 mmol, yield=5%).

¹H NMR (600 MHz, CDCl₃) δppm=−2.76 (s, 2H, H1); 7.76 (m, 12H, H4 and H5); 8.22 (d, J=7Hz, 8H, H3); 8.85 (s, 8H, H2)

¹³C NMR (150 MHz, CDCl3) δppm=120.28 (Cq); 126.83 (C4); 127.85 (C5); 134.70 (C3); 142.32 (Cq)

ESI-MS m/z calc. for [C₄₄H₃₀N₄]=614.3; found: [M+H⁺]²⁺=615.3, [M+2H⁺]²⁺=308.1

4′-Amino-5,10,15,20-Tetraphenylporphyrin (Compound 29)

TPPH (906 mg, 1.5 mmol, 1.0 eq.) was dissolved in 70 mL of TFA, then sodium nitrite (181 mg, 2.6 mmol, 1.8 eq.) was added. The solution was stirred at room temperature for exactly 3 min. The reaction mixture was then quenched with 200 mL of water. The aqueous solution was extracted with DCM. The organic layer was washed with saturated aqueous NaHCO₃ solution, dried over anhydrous Na₂SO₄, filtered and evaporated in vacuo to give a purple solid. The latter was dissolved without purification in 50 ml of concentrated HCl. Tin (II) chloride dihydrate (3.88 g, 17.0 mmol, 11.5 eq.) was added to this solution. The mixture was stirred at 65° C. for 2 h. The reaction mixture was quenched with 100 mL of cold water. The aqueous solution was basified to pH 14 by addition of ammonium hydroxide solution and extracted with DCM until colorless. The combined organic layer was dried over anhydrous Na₂SO₄, filtered and evaporated in vacuo to give a purple solid, which was purified by column chromatography (silica, DCM) to give the desired TPP-NH 2 compound as a powder. purple (443 mg, 7.05.10⁻⁴ moles, yield=51%).

¹H NMR (600 MHz, CDCl₃) δppm=−2.75 (s, 2H, H1); 7.08 (d, J=8.9 Hz, 2H, H7 or H8); 7.78-7.73 (m, 9H, H4 and H5); 8.01 (d, J=8.3 Hz, 2H, H7 or H8); 8.22 (d, J=6Hz, 6H, H3); 8.83 (s, 6H, H2); 8.94 (d, J=4.45 Hz, 2H, H6)

¹³C NMR (150 MHz, CDCl₃) δppm=113.61 (C8 or C7); 126.80 (C4); 127.50 (C5); 134.71 (C3); 135.85 (C7 or C8)

ESI-MS m/z calc. for [C₄₄H₃₁N₅]=629.3; found: [M+H⁺]⁺=630.3; [M+2H]²⁺=315.6.

### 5. General Procedure for functionalization of PAMAM-G3-NFI₂ (G3P-(NH₂)₃₂) Dendrimers

In a flask are dissolved n equivalents of 9 (4 eq./NH₂ under argon for a total functionalization i.e. 128 eq. or 0.5 eq./NH₂ for hemifunctionalization or 16 eq.), N eq. HBTU and n eq. of DIPEA in 10 mL of dry, degassed DMF. The solution is stirred at room temperature for 15 min. Meanwhile, 1 eq. of G3P-(NH₂)₃₂ (typically, 0.100 mL of a 12.44% solution of G3P-(NH₂)₃₂ in MeOH, i.e. 1.8 μmol of dendrimer) are dissolved in 10 mL of anhydrous DMF. Next, argon was bubbled in the dendrimer solution for 5 min before adding it to the activated azabodipy solution. The reaction is allowed to stir overnight at room temperature in the dark. The next day, the solution is concentrated under reduced pressure to evaporate the maximum of DMF and DIPEA. The pasty blue residue is then dissolved in high quality DMSO for analysis. and placed in a bag of dialysis membrane (10 kDa) sealed with clips. The dialysis rod is immersed in DMSO p.a. in a 500 ml Erlenmeyer flask and stirred gently in the dark. During dialysis, the dialysis DMSO is changed every hour on the first day, then every half day on the following days. After one week of dialysis, no blue coloration is observed in the dialysis solvent, the functionalized dendrimer solution is recovered inside the flange and the maximum of DMSO evaporated by evaporation under vacuum. The functionalized dendrimer G3P-(Bodipy1)ₙ (n=16,32) is recovered as an intense blue powder in quantitative yield.

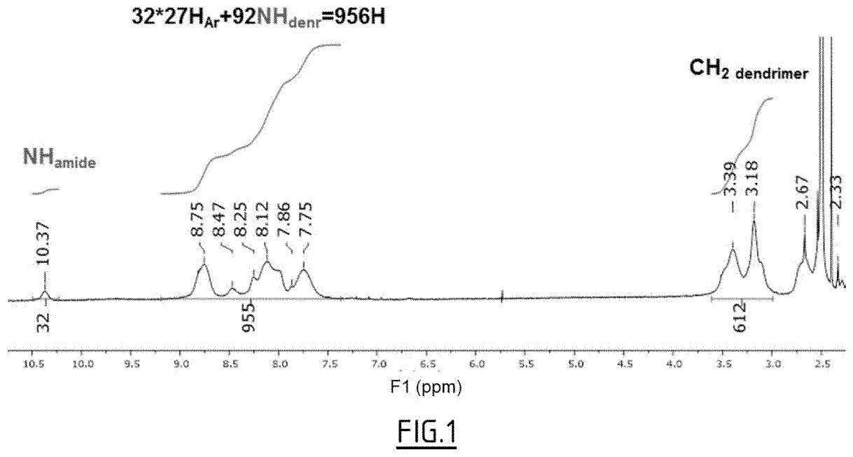

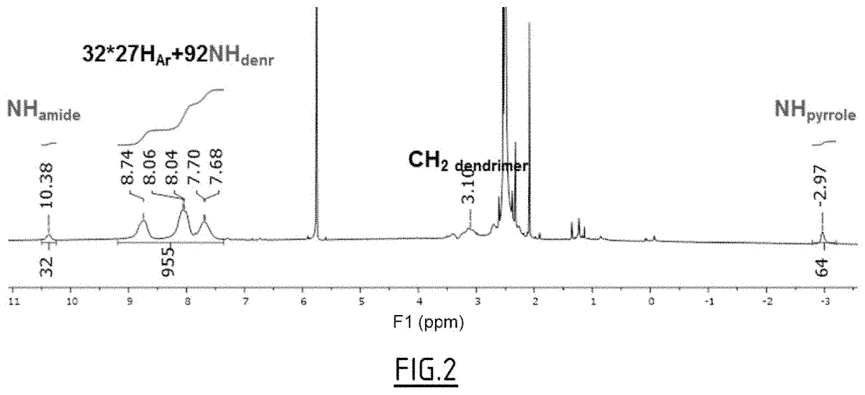

In order to conjugate the porphyrin derivative (29) to the PAMAM-G3 dendrimer by amide bonds, the thirty-two monoamide succinic acids on the surface of the dendrimer are activated by a mixture of HATU and DIPEA in DMF. The reaction mixture is stirred at room temperature for 30 min, then the monoamino porphyrin (29) is added. The reaction mixture is stirred for 3 days. The final product is purified by dialysis in DMSO using a MWCO 10kDa cut-off membrane. The product (G3-(TPP)₃₂) is characterized by ¹H NMR in DMSO-d₆ and makes it possible to demonstrate the complete substitution of the dendrimer by thirty-two porphyrins (FIG. 1, 2).

### 6. Preparation of Dendrimers According to the Invention

### 6.1. Compound G3P-(alkyne)₃₂

Hexynoic acid (428 mg, 3.8.10⁻⁴ mol, 40.0 eq.), hydrochloric N-(3-dimethylaminopropyl)-N′-ethylcarbodiimide (592 mg, 3.8.10⁻⁴ mol, 40.0 eq.), HOBT (516 mg, 3.8.10⁻⁴ mol, 40.0 eq.) and DIPEA are solubilized in 40 mL distilled DMF. The solution is stirred under an inert atmosphere for 30 min. To this solution is added a solution of G3P-(NH₂)₃₂ (660 mg, 9.5 10⁻⁵ mol, 1.0 eq.). The reaction mixture is stirred under an inert atmosphere at 25° C. for 48 h. At the end of the reaction, the DMF is evaporated under reduced pressure. The crude reaction product is purified by dialysis in DMSO for 24 hours. The solvent is then evaporated under reduced pressure. The compound G3P-(alkyne)₃₂ is obtained in the form of an orange oil.

¹H NMR (600MHz, DMSO-d₆): 7.97 (brs), 7.87 (brs), 312-3.07 (m), 2.72-2.69 (m), 2.47 (brs), 2.22-2.20 (brs), 2.16-2.12 (m), 1.64 (q).

¹³C NMR (MHz, DMSO-d6): 172.20, 171.89, 171.64, 84.31, 71.56, 52.18, 49.62, 40.43, 39.24, 36.86, 34.41, 33.06, 24.37, 17.58.

### 6.2. General Procedure for the Synthesis of G3P-(L2)₃₂

G3P-(alkyne)₃₂ (20 mg, 2.0.10⁻⁶ mol, 1.0 eq.) and the chromophore bearing an azide function (compound Perylene) (8.0.10⁻⁵ mol, 40 eq.) are solubilized in 1 ml of distilled DMF. The solution is degassed under argon for 20 min. To this solution is then added a solution of CuSO₄.5H₂O and sodium ascorbate in a mixture of H₂O/DMF (1 ml, 1/1, v/v). The reaction mixture is stirred under an inert atmosphere at room temperature for 24 h. At the end of the reaction, the solvents are evaporated under reduced pressure and an EDTA solution is added to the reaction crude. The solution is stirred for 12 h and then dialyzed in water for 24 h. The solvent is then evaporated under reduced pressure and the residue obtained is dialysed in DMSO for 24 h. At the end of the purification, the DMSO is evaporated under reduced pressure and the desired product (G3P-(Perylène)₃₂) is obtained in the form of a powder.

With compound 25 as nitrogen chromophore:

(G3P-(Perylène)₃₂) ¹H NMR (600 MHz, CDCl₃): 8.47 (s), 8.42 (d), 8.06-7.99 (brs), 7.725 (t), 7.54 (brs), 7.17-7.09 (brs), 4.85 (brs), 4.57 (brs), 4.30 (brs), 4.19-4.14 (brm), 3.47 (brs), 3.29 (brs), 2.34 (brs), 2.24 (brs), 1.89 (brs), 1.69 (brs), 1.40 (brs), 1.31 (brs) 0.85-0.81 (m).

### 6.3. General Procedure for the Synthesis of a G3P-(Alkyne)ₓ-(L2)_{y} Dendrimer

G3P-(Alkyne)₃₂ (20 mg, 2.0 10⁻⁶ mol, 1.0 eq.) and the azoture chromophore (1.6.10⁻⁵ mol, 8.0 eq.) are solubilized in 1 mL DMF. The solution is degassed under argon for 20 min. A solution of CuSO₄.5H₂O (7.10-6 mol, 3.5 eq.) and sodium ascorbate (7.10⁻⁶ mol, 3.5 eq.) (1 mL, DMF/H₂O (1/1, v/v)) is then added. The reaction mixture is stirred under argon at 25° C. for 12 h. At the end of the reaction, the solvents are evaporated under reduced pressure. The reaction crude is triturated in an aqueous solution of EDTA for 5 h and then dialyzed in water (MW 2 kDa). At the end of the dialysis, the solvents are evaporated under vacuum and the compound G3P-(Alkyne)ₓ-(L2)_{y} (x=22-24, y=8-10).

With compound 25 as azoture chromophore,

G3P-(Alkyne)ₓ-(Perylene)_{y} (x=22-24, y=8-10)

¹H NMR (600 MHz, CDCl₃): 8.16-8.09 (brm), 7.48 (brs), 7.19 (brs), 4.32 (brs), 4.18-4.13 (brs), 3.53 (brs), 3.33 (brs), 2.70 (brs), 2.58 (brs), 2.26 (brs), 1.94 (brs), 1.84 (brs), 1.74 (brs), 1.44 (brm), 1.34 (brs m), 0.89 (brs)

With cyanine as azoture chromophore, G3P-(Alkyne)ₓ-(Cyanine)_{y} (x=22-24, y=8-10)

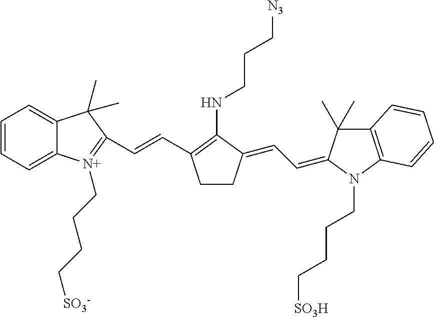

¹H NMR (600 MHz, DSMO-d₆): 7.95 (brs), 7.88 (brs), 7.74 (brs), 7.42 (brm), 7.27 (brm), 7.17 (brm). 7.04 (brm), 5.64 (m), 4.51 (brs), 3.93 (brs), 3.77 (brs), 2.75 (brm), 2.68 (brs) , 2.20 (brs), 2.14 (brs), 1.76 (brs), 1.65 (brs), 1.57 (brs).

With compound 18 as nitrogen chromophore

G3P-(Alkyne)ₓ-(Bodipy2)_{y} (x=22-24, y=8-10)

¹H NMR (600 MHz, DSMO-d6): 7.95 (brs), 7.89 (brs), 7.55-7.41 (m), 7.12 (brm), 6.92 (brm), 4.93-4.73 (m), 4.37 (brs), 3.51 (brs), 3.39 (brs), 3.08 (brs), 2.75 (brs), 2.67 (brs), 2.21 (brs), 2.15 (brs), 1.76 (brs), 1.65 (t).

### 6.4. General Procedure for the Synthesis of a G3P-(R2)ₓ-(L2)_{y} Dendrimer

The dendrimer G3P-(Alkyne)ₓ-(L2)_{y} (x=22-24, y=8-10) is solubilized in 1 mL of DMF. To this solution is added compound 27, the nitrogenous hydrosolubilizing group (4.0.10⁻⁵ mol, 20.0 eq.). The solution is degassed under argon for 20 min. A solution of CuSO₄.5H₂O (2.0.10⁻⁵ mol, 10.0 eq.) and sodium ascorbate (2.0.10⁻⁵ mol, 10.0 eq.) (1 mL, DMF/H₂O (1/1, v/v)) is then added. The reaction mixture is stirred under argon at 25° C. for 12 h. At the end of the reaction, the solvents are evaporated under reduced pressure. The reaction crude is triturated in an aqueous solution of EDTA for 5 h and then dialyzed in water (MW 2 kDa). At the end of the dialysis, the solvents are evaporated under reduced pressure and the residue is solubilized in DMSO and then dialysed in DMSO (MW 10 kDa). At the end of the dialysis, the DMSO is evaporated under reduced pressure. The desired product G3P-(R2)ₓ-(L2)_{y} (x=22-24, y=8-10) is obtained as a powder.

With compound 25 as azide chromophore and compound 27 as nitrogenous water-solubilising group, G3P-(PEG)ₓ (Perylene)_{y} (x=22-24, y=8-10)

¹H NMR (600 MHz, CDCl₃): 8.16-80.9 (brm), 7.45 (brs), 7.56 (s), 7.49 (brs), 7.13 (brs), 4.49 (s), 4.33 (brs), 4.19-4.14 (brm), 3.84 (s), 3.61 (s), 3.61 (s), 3.36 (s), 3.32 (brs), 2.71 (s), 2.35 (brs), 2.25 (brs), 1.95 (brs), 1.85 (brs), 1.74 (brs), 1 , 44 (brs), 1.34 (brs).

With compound 18 as azide chromophore and compound 27 as nitrogenous hydrosolubilizing group, G3P-(PEG)ₓ-(Bodipy2)_{y} (x=22-24, y=8-10)

¹H NMR (600 MHz, DSMO-d₆): 7.96 (brs), 7.86 (brs), 7.81 (s), 7.47-7.36 (brsm), 7.00 (brm), 6.70-6.65 (brm), 5.16 (brs), 4.89 (brs), 4.45 (s), 3.78 (s), 3.78 (s), 3.50 (s), 3.46 (brs), 3.39 (brs), 3.34 (brs), 3.31 (brs), 3.08 (brs), 2.78 (brs), 2.65 (brs), 2.59-2.56 (brm), 2.42 (brs), 2.19 (brs), 2.11 (t), 1.79 (t).

### Example 2: Synthesis of Complexes According to the Invention

To prepare dendrimer complexes containing the lanthanide cations, a dendrimer solution in DMSO was treated with 8 eq. of lanthanide nitrate for 7 days at room temperature.

### Example 3: Properties of Complexes According to the Invention

Photophysical Properties of Aza-Bodipy-Based Molecules

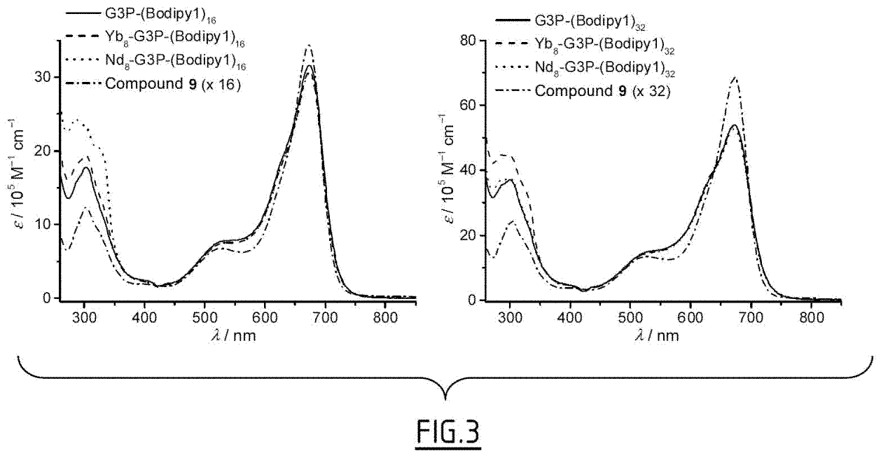

The absorption spectrum of the Bodipy chromophore (compound 9) measured in a DMSO solution has a broad band of up to 770 nm with a low energy main band at 672 nm (ϵ=2.1.10⁵ M⁻¹ cm⁻¹), FIG. 3). Attaching this chromophore to the surface of the G3 PAMAM dendrimer (G3P-(Bodipy1)ₙ, n=16, 32) as well as the encapsulation of Ln³⁺ ions within its branches do not affect the position and the shape of low energy absorption bands. On the other hand, the molar coefficient of absorption increases with the number of Bodipy chromophore units attached.

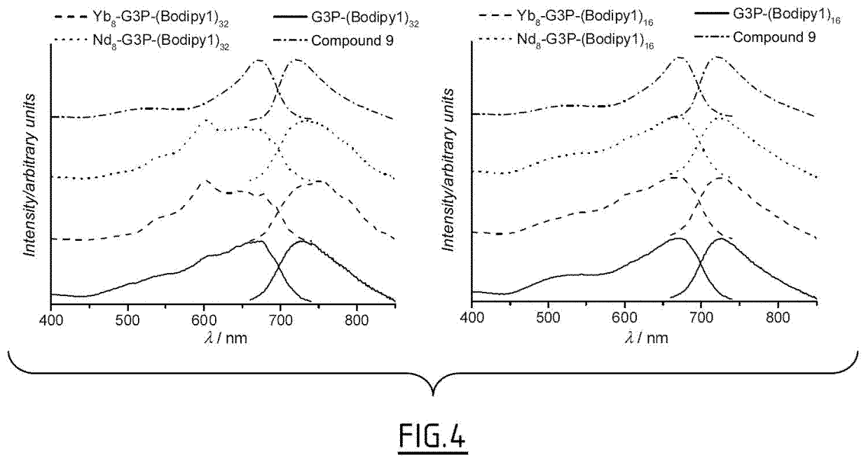

Under excitation centered at 650 nm, corresponding to the maximum absorption of the chromophore, the compound 9 as well as the dendrimers (Ln₈-)G3P-(Bodipy1)ₙ (n=16, 32, Ln=Yb³⁺, Nd³⁺) show broad, very similar emission bands with a maximum at ˜735 nm from energy levels centered on the aza-Bodipy chromophore (FIG. 4). (Ln₈-)G3P-(Bodipy1)₃₂ (Ln=Yb³⁺, Nd³⁺) shows a slight broadening of the emission bands as well as a shift towards wavelengths at lower energies. The forms of the excitation spectra measured under observation of the emission at 760 nm correspond to those of the absorption spectra (FIG. 4 vs. 3). The characteristic luminescence in the form of narrow bands of Yb³⁺ or Nd³⁺ ions could not be observed in the near infrared under excitation of the chromophore. This result can be explained by an insufficient capacity of the Bodipy chromophore to sensitize the lanthanides tested or an unbalanced ratio between the number of chromophore groups and the number of Ln3+ which could induce the masking of the characteristic signals of Nd³⁺ or Yb³⁺ under a broad band of emission from organic groups.

The absolute quantum yields of the chromophore (compound 9) and dendrimers (Ln₈-)G3P-(Bodipy1)ₙ (n=16, 32; Ln=Yb³⁺, Nd³⁺) are summarized in Table 1. Significant decreases (6 and 3.4 times) in quantum emission efficiencies from the aza-BODIPY-COOH chromophoric moieties for the functionalized dendrimers, G3P-(aza-BODIPY)₃₂ and G3P-(aza-BODIPY)₁₆, respectively. Such an effect is probably caused by the self-quenching of the chromophores located on the PAMAM G3P dendrimer. The presence of Ln³⁺ within the branches of G3P-(aza-BODIPY)₁₆ does not affect the quantum efficiency values which remain the same taking into account the experimental error. On the other hand, the Q values are increased by 1.7 and 1.4 times for G3P-(Bodipy1)₃₂ during the encapsulation of the lanthanide cations Yb³⁺ and Nd³⁺, respectively. This result can be explained by the formation of different conformations of dendrimers in Ln₈-G3P-(Bodipy)₃₂ with reduced interactions between chromophore units thus modulating self-extinction.

TABLE 1
Quantum yields (Q) of a 10 μM concentration solution
of compound 9 and (Ln₈−)G3P-(Bodipy1)ₙ (n = 16, 32; Ln =
Yb³⁺, Nd³⁺) measured in DMSO at room temperature

| Compound | Q (%) |
|---|---|
| Compound 9 | 8.04(8) |
| G3P-(Bodipy1)₃₂ | 1.33(3) |
| Yb₈-G3P-(Bodipy1)₃₂ | 2.25(3) |
| Nd₈-G3P-(Bodipy1)₃₂ | 1.85(1) |
| G3P-(Bodipy1)₁₆ | 2.40(3) |
| Yb₈-G3P-(Bodipy1)₁₆ | 2.53(7) |
| Nd₈-G3P-(Bodipy1)₁₆ | 2.27(3) |

^{a}The values 2σ are indicated in parentheses. Experimental errors: Q, ±10%. Quantum yields (Q) measured under excitation at 650 nm by collecting the emission in the range of 660 to 850 nm.

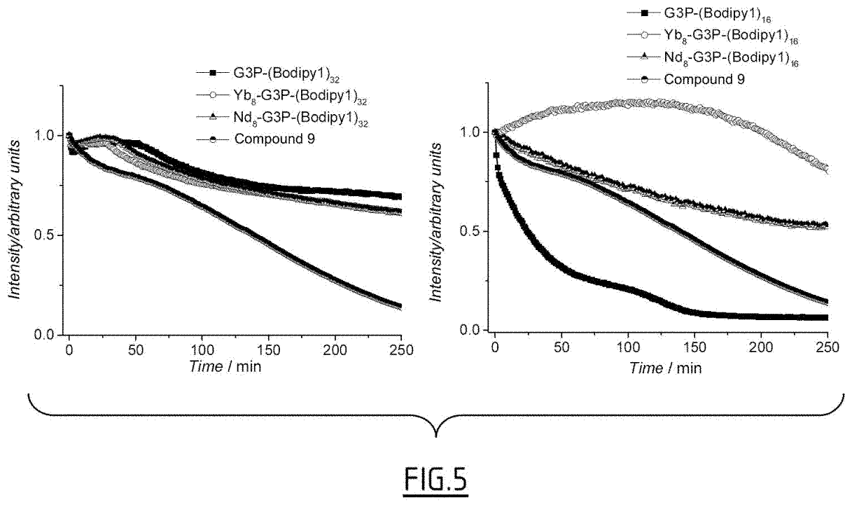

To test the photostability of the system, the chromophore (compound 9) and the dendrimers (Ln₈-)G3P-(Bodipy1)ₙ (n=16, 32; Ln =Yb³⁺, Nd³⁺) were continuously illuminated with 670 lumen. nm and the emission signal was monitored at 735 nm. The results (FIG. 5) revealed significantly different behaviors for (Ln₈-)G3P-(Bodipy1)₃₂ and for (Ln₈-)G3P-(Bodipy1)₁₆. The fully functionalized dendrimers and their complexes formed with Yb³⁺ and Nd³⁺ exhibit very similar photostability with a decrease of only 28-34% in the initial luminescence intensity after 3 h of illumination. Under the same conditions, the partially functionalized dendrimer, G3P-(Bodipy1)₁₆ completely loses its emission signal (FIG. 5, right). However, the photostability of G3P-(Bodipy1)₁₆ could be improved by the incorporation of Ln³⁺. In addition, it turned out to be dependent on the nature of the lanthanides. Thus, the intensity of Nd₈-G3P-(Bodipy1)₁₆ is decreased by 40% after 3 h of illumination while the emission signal of Yb₈-G3P-(Bodipy1)₁₆ after a slight increase of the emission signal returns at its initial intensity value after 200 min of treatment. As for the initial chromophore, compound 9 (FIG. 5) loses 75% of its luminescence intensity after 3 hours of illumination. Therefore, in general, the attachment of the chromophore moieties to the dendrimer structure and the presence of Ln³⁺ can be advantageously used to provide and enhance the photostability of the probe.

### Cytotoxicity of Aza-Bodipy-Based Molecules

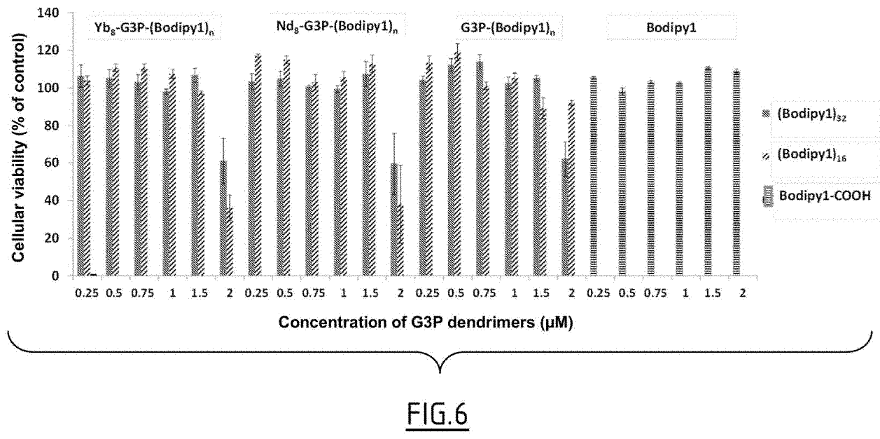

In order to determine the potential toxicity of the described dendrimers, an Alamar Blue test was performed on the HeLa ovarian carcinoma human cell line (FIG. 6). Different concentrations of chromophores (compound 9) and dendrimers (Ln₈-)G3P-(Bodipy1)ₙ (n=16, 32; Ln=Yb³⁺, Nd³⁺) were tested. The chromophore does not induce toxicity up to the concentration of 2 μM with ˜100% cell viability observed.

### Confocal Fluorescence Microscopy of Bodipy-Based Compounds

Confocal microscopy experiments were performed to confirm the intracellular localization of the dendrimers (FIG. 7). For this purpose, HeLa cells were incubated for 30 min with a concentration solution of 1.5 μM, which corresponds to ˜89-100% of cell viability. The fluorescence of the dendrimers was observed using a 633 nm laser as the excitation source and the emission of aza-BODIPY was collected between 650 and 800 nm with an optical cut of 1 μm in the z axis. The same laser power was used for the different experiments to compare the signal intensity variations between different dendrimers. The most intense signal was observed for Yb₈-G3P-(aza-BODIPY)₁₆ whereas the images acquired for HeLa cells incubated with Nd₈-G3P-(aza-BODIPY)₁₆ have a relatively less intense intensity (Fig. top: (A) and (B)). In the case of the G3P-(aza-BODIPY)₁₆ and (Ln₈-)G3P-(aza-BODIPY)₃₂ dendrimers the observed signal was relatively weak with the same laser settings (power set at 6%) (FIG. 7, top: (C) and bottom: (A) and (B)). The HeLa cells incubated with aza-BODIPY-COOH show a signal of lower intensity than that observed for Yb₈-G3P-(aza-BODIPY)₁₆(Fig. 7, top: (A) vs. (D)), despite a quantum yield of aza-BODIPY-COOH 3.4 times higher than that of Yb₈-G3P-(aza-BODIPY)₁₆ (Table 2).

Differences in signal intensity in fully or partially functionalized dendrimer cells, with or without encapsulated Ln³⁺, can be attributed to the importance of cell internalization, which varies with differences in solubility in the cell. water or conformations of macromolecules. However, in all cases, the intracellular signal distribution was observed in the cytoplasm and more specifically in the lysosomes, with a particularly strong signal for the Yb₈-G3P-(aza-BODIPY)₁₆ dendrimer. In addition, a formation of several long and fine filipodia, structures derived from F-actin-rich plasma membranes were observed for the various dendrimers studied and the aza-BODIPY-COOH molecules (FIG. 7, yellow arrows). Experiments will be needed to confirm this specific labeling, in particular colocalization experiments with commercially available probes of AlexaFluor types conjugated to a phalloidin (peptide specific for actin labeling).

It should be noted that under these experimental conditions used, no autofluorescence signal was detected, which again demonstrates the advantages of using probes with excitation and emission wavelengths in the biological imaging window.

### Flow Cytometry Experiments with Bodipy-Based Molecules Flow

Cytometry has been used to quantify cell internalization of dendrimers and to better understand signal intensity differences observed in confocal microscopy. The mechanisms of passive (non-dependent energy) and active (energy dependent) internalization of cells have been studied. Active transport was inhibited with sodium azide (NaN₃), while incubation at 4° C. inhibited active and passive transport pathways by increasing the plasma membrane stiffness of HeLa cells (S. Vranic, N. Boggetto, V. Contremoulins, S. Mornet, N. Reinhardt, F. Marano, A. Baeza-Squiban, S. Boland, Deciphering the mechanisms of cellular uptake of nanoparticles by accurate evaluation of internalization for cytometry, Particle and Fiber Toxicology, 10 (2013) 1-16).

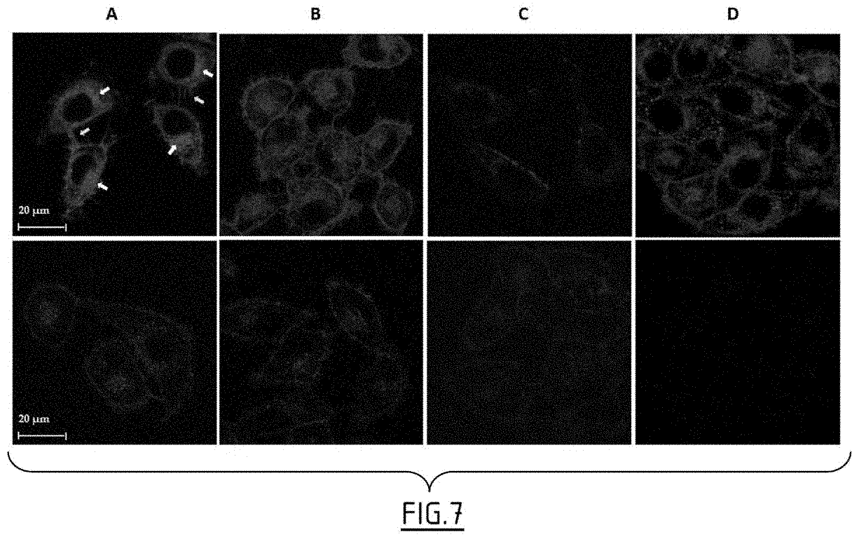

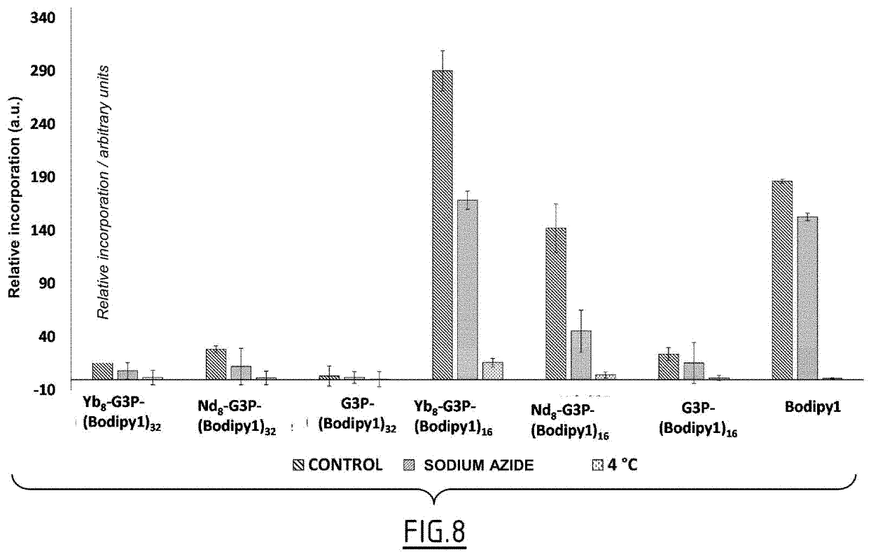

The results obtained make it possible to conclude without ambiguity that the partially functionalized dendrimers (Ln₈-)G3P-(Bodipy1)₁₆ (Ln=Yb³⁺, Nd³⁺) are significantly more internalized by the cells compared to fully functionalized dendrimers (Ln₈-)G3P-(Bodipy1)₃₂ (Ln=Yb³⁺, Nd³⁺) (FIG. 8). However, for both types of dendrimers, internalization is consistently higher for those containing Yb³⁺ than for those containing Nd³⁺. In fact, the incubation with the Yb₈G3P-(Bodipy1)₁₆ dendrimer shows a greater cellular internalization than the Nd₈G3P-(Bodipy1)₁₆. dendrimer. These results can be explained by the better solubility and consequently a lower aggregation in the cell medium of the partially functionalized dendrimers as well as a conformation more favorable to the internalization of the dendrimers having encapsulated Ln³⁺ (particularly for Ln=Yb³⁺). For the chromophore compound 9, a relatively strong emission signal was observed and corresponding to 65% internalization of Yb₈G3P-(Bodipy1)₁₆. On the other hand, the internalization of the Nd₈-G3P-(Bodipy1)₁₆ dendrimer weaker than that of the Yb₈-G3P-(Bodipy1)₁₆ dendrimer is compensated by a higher quantum yield (Table 2) making it possible to observe intensities of comparable emission in confocal microscopy (FIG. 7, top (A), (B) vs (D)). Dendrimers enter cells through active and passive transport pathways (FIG. 8). The results corresponding to the inhibition of cell internalization are shown in Table 2. The relative percentages of active or passive internalization vary according to the number of Bodipy chromophore and the presence of Ln³⁺. The chromophore compound 9 shows 19% internalization by active transport. However, further studies are needed to clarify the exact mechanisms of active transportation. Endocytosis is the primary active mechanism for entry into the cell (B. D. Grant, J. G. Donaldson, Pathways and Mechanisms of Endocytic Recycling, Nature Reviews Molecular Cell Biology, 10 (2009) 597-608, L. Kou, J. Sun , Y. Zhai, Z. He, The endocytosis and intracellular fate of nanomedicines: implications for rational design, Asian Journal of Pharmaceutical Sciences, 8 (2013) 1-10, D. Vercauteren, R. E. Vandenbroucke, A. T. Jones, J. Rejman, J. Demeester, S. C. De Smedt, N. N. Sanders, K. Braeckmans, Molecular Therapy, 18 (2010) 561-569, D. A. Kuhn, D. Vanhecke, B. K. Braeckmans, The use of inhibitors to study endocytic pathways of genes Michen, F. Blank, P. Gehr, A. Petri-Fink, B. Rothen-Rutishauser, Different endocytotic uptake mechanisms for nanoparticles in epithelial cells and macrophages, Beilstein Journal of Nanotechnology, 5 (2014) 1625-1636), flow cytometry measurements can be performed after inhibition of the different pathways endocytosis with appropriate molecules such as: (D. Dutta, J. G. Donaldson, Research for inhibitors of endocytosis: uniquely intended and unintended consequences, Cell Logist, 2 (2012) 203-208) Monodansylcadaverine (H. T. McMahon, E. Boucrot, Molecular mechanism and physiological functions of clathrin-mediated endocytosis, Nature Reviews Molecular Cell Biology, 12 (2011) 517-533), Filipin III (J. E. Schnitzer, P. Oh, E. Pinney, J. Allard, Filipin-sensitive caveolae-mediated endothelium-reduced transcytosis transport, scavenger endocytosis, and capillary-permeability of selected macromolecules, Journal of Cell Biology, 127 (1994) 1217-1232) or Amiloride (S. Gold, P. Monaghan, P. Mertens, T. Jackson, A clathrin independent macropinocytosis-like entry mechanism used by bluetongue virus-1 during infection of BHK cells, PLoS One, 5 (2010) e11360), respective inhibitors of clathrin- and caveolin-dependent endocytosis or micropinocytosis.

TABLE 2
Percentage of Cellular Inhibition of Internalization by Active
and Passive Transport Mechanism of Compounds 9 and Dendrimers
(Ln₈−)G3P-(Bodipy1)ₙ (n = 16, 32; Ln = Yb³⁺, Nd³⁺)
(HeLa cells). The values were obtained by flow cytometry.

Cellular internalization

| Compounds | Active | Passive^{a} |
|---|---|---|
| Yb₈-G3P-(Bodipy1)₁₆ | 42 ± 8 | 53 ± 4 |
| Nd₈-G3P-(Bodipy1)₁₆ | 68 ± 20 | 29 ± 3 |
| G3P-(Bodipy1)₁₆ | 35 ± 19 | 57 ± 2 |
| Yb₈-G3P-(Bodipy1)₃₂ | 72 ± 8 | 22 ± 7 |
| Nd₈-G3P-(Bodipy1)₃₂ | 57 ± 17 | 38 ± 6 |
| G3P-(Bodipy1)₃₂ | 40 ± 5 | 51 ± 7 |
| Composé\acute{ }9 | 18 ± 4 | 81 ± 1 |

^{a}The percentages of inhibition of the passive mechanisms are presented as the percentage difference between the internalization inhibited at 4° C. and that inhibited with sodium azide.

Epifluorescence Microscopy with Bodipy-Based Molecules

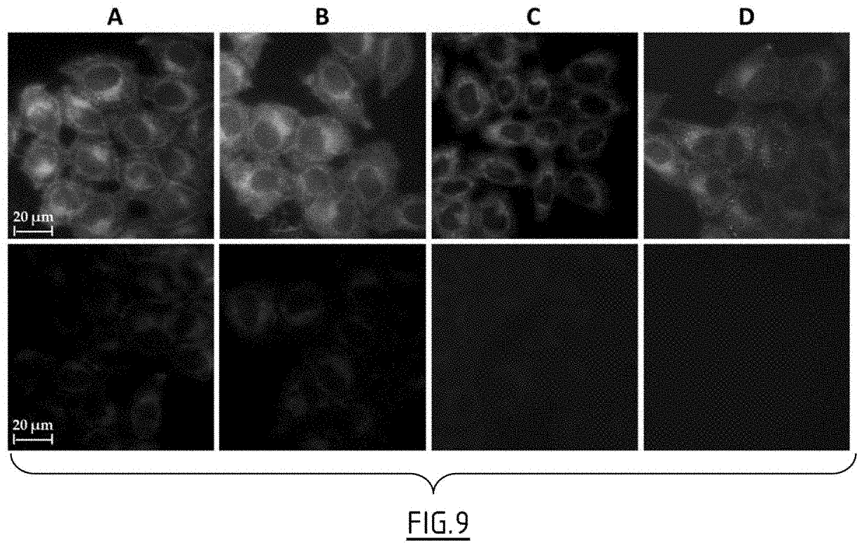

The detection capacity of the signal emitted in the near infrared by the compounds 9 and the dendrimers (Ln₈-) G3P-(Bodipy1)ₙ (n=16, 32; Ln=Yb³⁺, Nd³⁺) was confirmed by epifluorescence microscopy after 30 min incubation of HeLa cells with a solution of 1.5 μM of corresponding dendrimer complex. Due to the near-infrared emission bandwidth of the aza-Bodipy chromophore, the signal was collected by epifluorescence with a long pass filter of 785 nm at emission and a 655±40 nm filter at excitation (FIG. 9). The results obtained are consistent with those obtained by confocal microscopy (FIG. 7). HeLa cells incubated with the dendrimer. Ln₈-G3P-(Bodipy1)₁₆ (FIG. 9, top: (A) and (B)) show the strongest emitted signal after 8 seconds of exposure. The images obtained for the other dendrimers show a weaker or non-detectable signal in the case of G3P-(Bodipy1)₃₂.

The results can again be explained by differences in cell internalization (Table 2) and emission intensities (Table 1) for the different dendrimers studied.

Photophysical Properties of Cyanine-Based Molecules

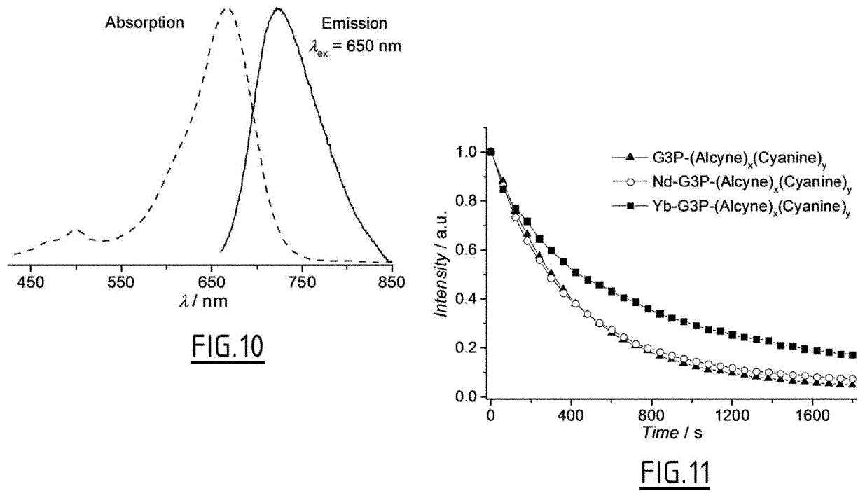

The dendrimer G3P-(Alkyne)ₓ-(Cyanine)_{y} (x=22-24, y=8-10) and its complexes with Nd and Yb show a broad absorption band centered at 665 nm (FIG. 10). The excitation on the bands centered on the ligand results in an intense broadband emission centered at 725 nm. No narrow emission bands characteristic of Nd or Yb could be observed in the near-infrared.

The continuous illumination of G3P-(Alkyne)ₓ-(Cyanine)_{y} (x=22-24, y=8-10) with a light centered at 665 nm makes it possible to estimate the photostability of the dendrimers which appears to be rather weak. (FIG. 11).

Epifluorescence Microscopy of Cyanine-Based Molecules

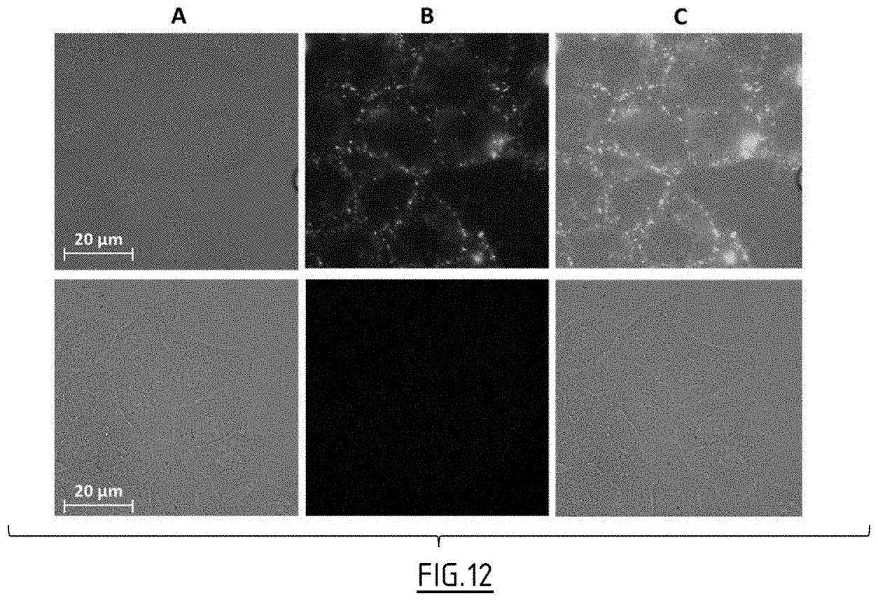

To establish the proof of principle that the emission of this dendrimer can be detected by epifluorescence microscopy after incubation with HeLa cells. A fluorescence signal was observed using a 655±40 nm excitation filter and a 750±50 nm emission filter (FIG. 12).

Photophysical Properties of Porphyrin-Based Molecules

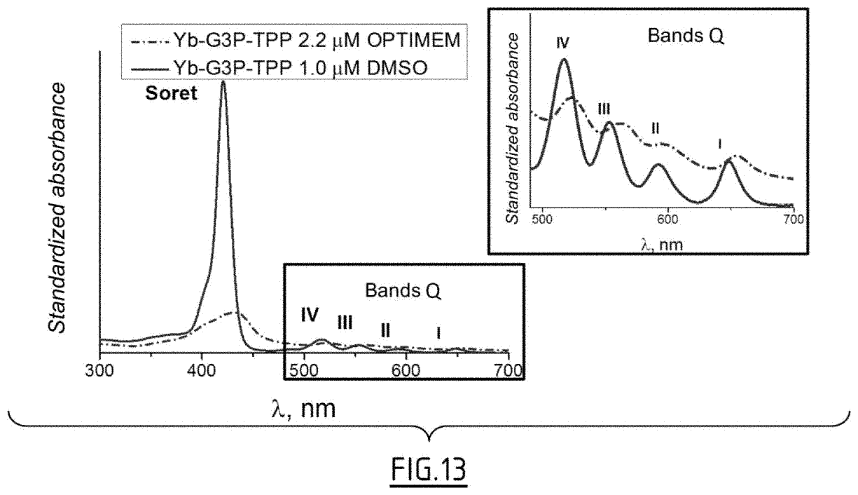

Absorption spectra of the Yb₈-G3P-(TPP)₃₂ complex were recorded in DMSO and in the DMSO/(Opti-MEM/FCS, 6: 94%) mixture (FIG. 13).

The absorption spectrum of Yb₈-G3P-(TPP)₃₂ measured in a solution of DMSO exhibited the typical porphyrin absorbance bands: the Soret band centered at 420 nm (ϵ˜5.9·10⁶ M⁻¹·cm⁻¹), the Q IV band centered at 517 nm (ϵ˜16.6·10⁴ M⁻¹·cm⁻¹), the Q III band centered at 553 nm (ϵ˜16.6·10⁴ M⁻¹·cm⁻¹), the Q II band centered at 592 nm (ϵ˜8.5·10⁴ M⁻¹·cm⁻¹), and the IQ band centered at 648 nm (ϵ˜9.1·10⁴ M⁻·cm⁻¹).

The bands present on the absorption spectrum of Yb₈-G3P-(TPP)₃₂ in the DMSO/(Opti-MEM: FCS) mixture are slightly redshifted by 3-11 nm. It should be noted here that in our case we have been limited by the low solubility of Yb₈-G3P-(TPP)₃₂ in the cell culture medium and, therefore, light scattering is important.

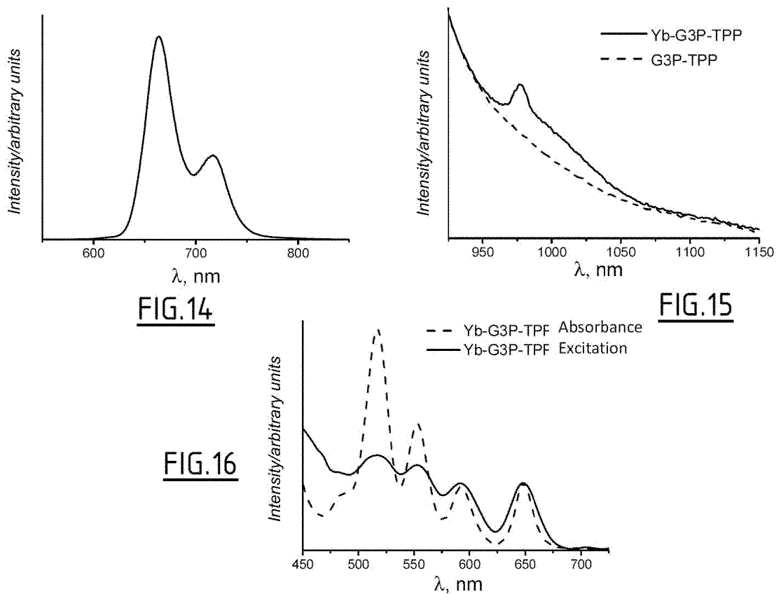

With excitation centered at 520 nm, Yb₈-G3P-(TPP)₃₂ shows a typical porphyrin emission: two bands in the visible region: centered at 664 nm and 717 nm, FIG. 14. A narrow band corresponding to the transition 2F²F_{5/2}→²F_{7/2} of the Yb³⁺ in the near-infrared was observed, FIG. 15. However, the signal seems to be superimposed on the residual emission of the tetraphenylporphyrin chromophore. This result can be explained by a low capacity of the chromophore to sensitize Yb³⁺ or an unbalanced ratio between the number of chromophore groups and that of lanthanide cations.

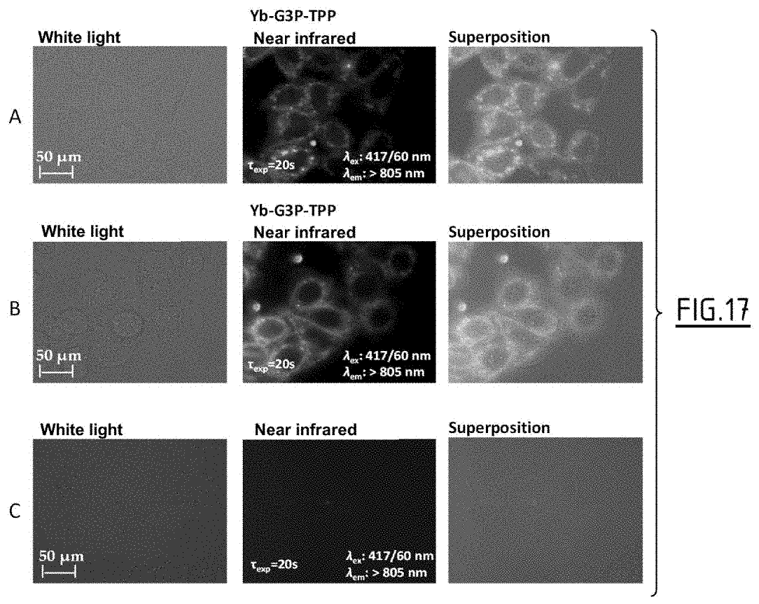

The excitation spectrum of Yb₈-G3-(TPP)₃₂ shows bands comparable to those observed on the absorption spectrum, thus indicating that the sensitization of Yb³⁺ occurs by the energy transfer of the tetraphenylporphyrin chromophore to the lanthanide cation, FIG. 16.

The quantum yield of the Yb³⁺ emission in the Yb₈-G3-(TPP)₃₂, complex, 37 μM in a DMSO solution could not be determined due to the too low intensity of the emission signal. Nevertheless, luminescence decay curves have been measured and can be deconvolved by bi-exponential functions with individual lifetime values of 68 (1) μs (48 (5)%) and 17.4 (1) μs (52 (5)%).

Epifluorescence Microscopy of Porphyrin-Based Molecules and Potential Activity in Photodynamic Therapy

The ability of the Yb₈-G3-(TPP)₃₂ complex to be used as an imaging agent in the near-infrared region has been confirmed by epifluorescence microscopy experiments. Intense signals were detected in the near-infrared excitation at 417 nm (60 nm bandpass filter) in HeLa cells incubated with the Yb₈-G3-(TPP)₃₂ complex (FIG. 17A). After 2 min of irradiation with a selected light with a bandpass filter centered at 417 nm, we observed the formation of vesicles. This result is a typical indication of an oxidative stress process resulting from the production of reactive oxygen species by Yb₈-G3-(TPP)₃₂ under light irradiation (FIG. 17B).

In the case of cells not incubated with Yb₈-G3-(TPP)₃₂, we did not observe autofluorescence in the near infrared region (FIG. 17C). The epifluorescence microscopy images show a certain level of aggregation of the Yb₈-G3-(TPP)₃₂ dendrimers under the biological conditions used.

What is claimed is:

1. Complex comprising at least one dendrimer and at least one lanthanide, wherein the dendrimer comprises a unit of formula (I) below: 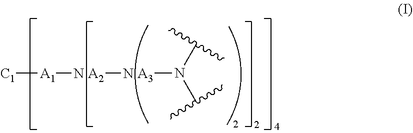
in which:
C₁ is a valence group 4 of formula >N—CH₂—CH₂—N<;
A₁, A₂ and A₃ are groups of formula —(CH₂)₂—C(O)—NH—(CH₂)₂-;said unit of formula (I) being connected covalently to at least one antenna which absorbs at a wavelength ranging from 500 nm to 900 nm, said antenna being chosen from the group consisting of: anthraquinones, cyanines, especially cyanines and cyanine 7, aza-BODIPY, perylenediimides, porphyrins, phenothiazine salts, and their derivatives.

2. Complex according to claim 1, wherein the antenna is connected to the unit of formula (I) covalently via at least one arm corresponding to the following formula:
-A₄-X-[A₅-Z]ᵢ-((A₆)ⱼ-Y)ₖ-
in which:
A₄ is a group of formula —(CH₂)₂—X′—(CH₂)₂—, X′ representing a —C(O)—NH— group or a —NH—C(O)— group, and being preferably a group of formula —(CH₂)₂—C(O)—NH—(CH₂)₂—;
X is a group of formula —NH—C(O)—;
i is an integer between 1 and 3;
j is 0 or 1;
k is 0 or 1;
A₅ and A₆ are chosen, independently of one another, from linear or branched (cyclo)alkylene radicals comprising from 1 to 12 carbon atoms;
Z is selected from —O—, —NH—, —S—, amide, ester, triazole, amine, ether, thioether, urea, thiourea, imine, oxyme, hydrazone, sulfonamide, carbamate, amidine, phosphoramidate, disulfide and sulfonyl; and
Y is selected from —O—, —NH—, —S—, alkylene, amide, ester, triazole, amine, ether, thioether, urea, thiourea, imine, oxyme, hydrazone, sulfonamide, carbamate, amidine, phosphoramidate , disulfide, and sulfonyl.

3. Complex according to claim 1, wherein the dendrimer has the following formula:
C₁-{A₁-N[A₂-N(A₃-N(A₄-R′)₂]₂}₄
in which:
C₁ is a valence group 4 of formula >N—CH₂—CH₂—N<;
A A₁, A₂, A₃ and A₄ are groups of formula —(CH₂)₂—C(O)—NH—(CH₂)₂—;
the radicals R′ are chosen, independently of one another, from the group consisting of:
groups of formula (1) below:
—NH—C(O)-[A₅-Z]ᵢ-((A₆)ⱼ-Y)ₖ-L
i is an integer between 1 and 3;
j is 0 or 1;
k is 0 or 1;
A₅ and A₆ are chosen, independently of one another, from linear or branched (cyclo)alkylene radicals comprising from 1 to 12 carbon atoms;
is selected from —O—, —NH—, —S—, amide, ester, triazole, amine, ether, thioether, urea, thiourea, imine, oxyme, hydrazone, sulfonamide, carbamate, amidine, phosphoramidate, disulfide and sulfonyl;
Y is selected from —O—, —NH—, —S—-, alkylene, amide, ester, triazole, amine, ether, thioether, urea, thiourea, imine, oxyme, hydrazone, sulfonamide, carbamate, amidine, phosphoramidate , disulfide, and sulfonyl;
L representing an antenna selected from the group consisting of: anthraquinones, cyanines, especially cyanines and cyanines 7, aza-BODIPY, perylenediimides, phenothiazine salts, and their derivatives; and
groups of formula (2) below:
—N H—C(O)-[A₅-Z]ᵢ-((A₆)ⱼ-Y)ₖ-L′
j, k, A₅, A₆, Z and Y being as defined above,
L′ representing a water-solubilising group or a targeting group, the water-solubilising group being preferably chosen from phosphates, sulphonates, sugars, and PEG chains, and the targeting group preferably being chosen from peptides, antibodies and oligonucleotides, biotin, sugars, and polysaccharides; and .
groups of formula (3)
—NH—C(O)-A₅-L″
A₅ being as defined above, and
L′ representing an alkyne group;
at least one of the R′ groups corresponding to the formula (1).

4. Complex according to claim 3, in which at least one of the R′ groups has the following formula (1′):
—NH—C(O)-A₅-Z-L
A₅ and Z being as defined in claim 2, and
L being as defined in claim 3.

5. Complex according to claim 4, in which at least one of the R′ groups has the following formula (1″): 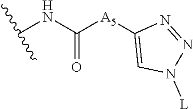
A₅ being chosen from linear or branched (cyclo)alkylene radicals comprising from 1 to 12 carbon atoms, and
L representing an antenna selected from the group consisting of: anthraquinones, cyanines, especially cyanines and cyanines 7, aza-BODIPY, perylenediimides, phenothiazine salts, and their derivatives; and
groups of formula (2) below:
—NH—C(O)-[A₅-Z]ᵢ-((A₆)ⱼ-Y)ₖ-L′
i is an integer between 1 and 3;
j is 0 or 1;
k is 0 or 1;
A₅ and A₆ are chosen, independently of one another, from linear or branched (cyclo)alkylene radicals comprising from 1 to 12 carbon atoms;
Z is selected from —O—, —NH—, —S—, amide, ester, triazole, amine, ether, thioether, urea, thiourea, imine, oxyme, hydrazone, sulfonamide, carbamate, amidine, phosphoramidate, disulfide and sulfonyl;
Y is selected from —O—, —NH—, —S—, alkylene, amide, ester, triazole, amine, ether, thioether, urea, thiourea, imine, oxyme, hydrazone, sulfonamide, carbamate, amidine, phosphoramidate , disulfide, and sulfonyl.

6. Complex according to claim 4, in which at least one of the R′ groups has the following formula (1′″):
—NH—C(O)-A₅-O-L
A₅ chosen from linear or branched (cyclo)alkylene radicals comprising from 1 to 12 carbon atoms, and
L representing an antenna selected from the group consisting of: anthraquinones, cyanines, especially cyanines and cyanines 7, aza-BODIPY, perylenediimides, phenothiazine salts, and their derivatives; and
groups of formula (2) below:
—NH—C(O)-[A₅-Z]ᵢ-((A₆)ⱼ-Y)ₖ-L′
i is an integer between 1 and 3;
j is 0 or 1;
k is 0 or 1;
A₅ and A₆ are chosen, independently of one another, from linear or branched (cyclo)alkylene radicals comprising from 1 to 12 carbon atoms;
Z is selected from —O—, —NH—, —S—, amide, ester, triazole, amine, ether, thioether, urea, thiourea, imine, oxyme, hydrazone, sulfonamide, carbamate, amidine, phosphoramidate, disulfide and sulfonyl;
Y is selected from —O—, —NH—, —S—, alkylene, amide, ester, triazole, amine, ether, thioether, urea, thiourea, imine, oxyme, hydrazone, sulfonamide, carbamate, amidine, phosphoramidate , disulfide, and sulfonyl.

7. Complex according to claim 1, wherein the antenna corresponds to one of the following formulas:

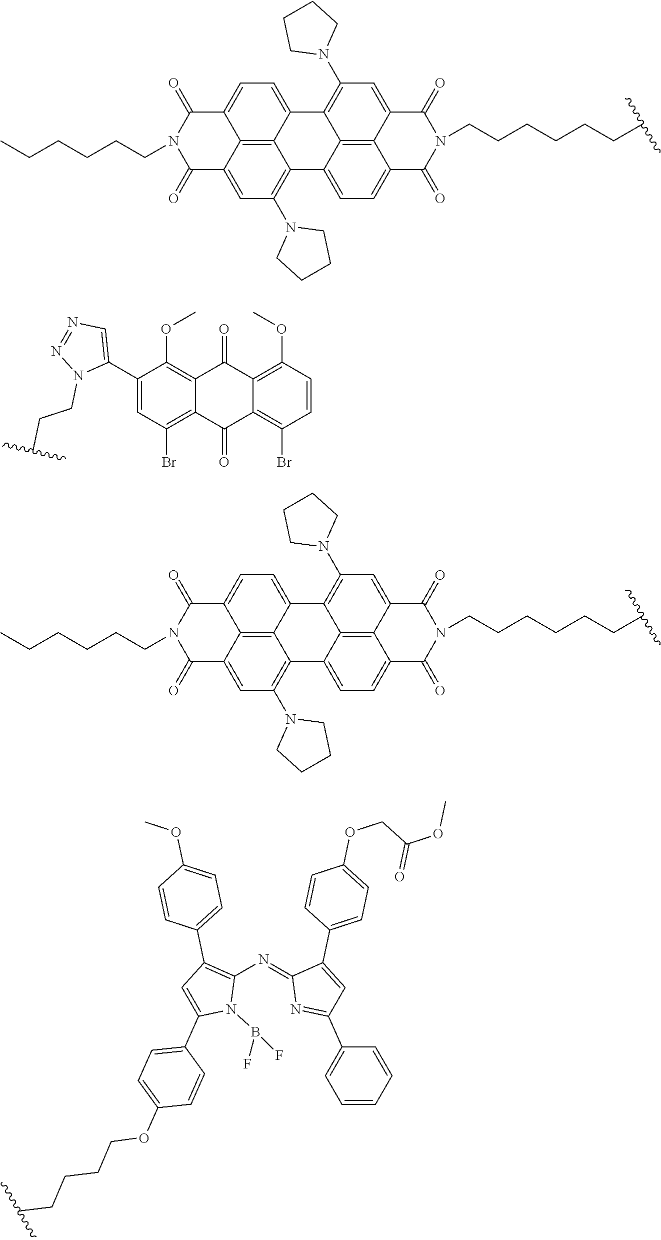

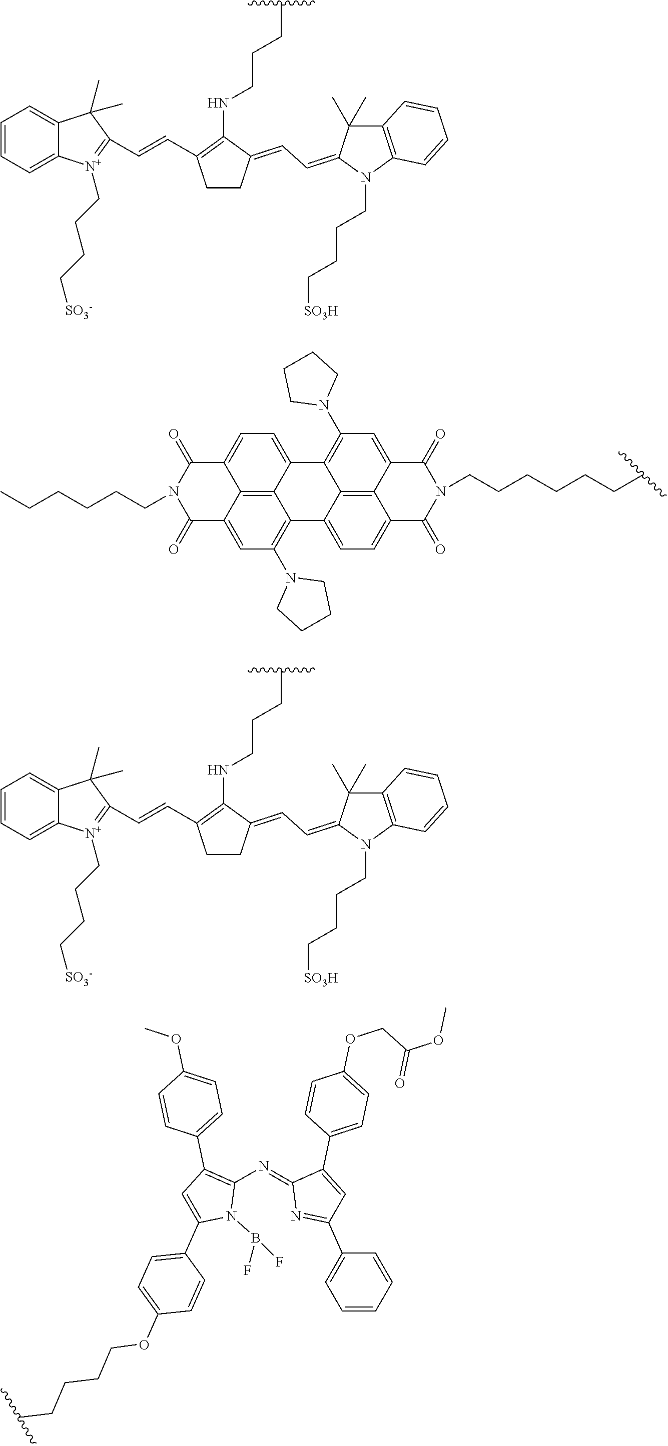

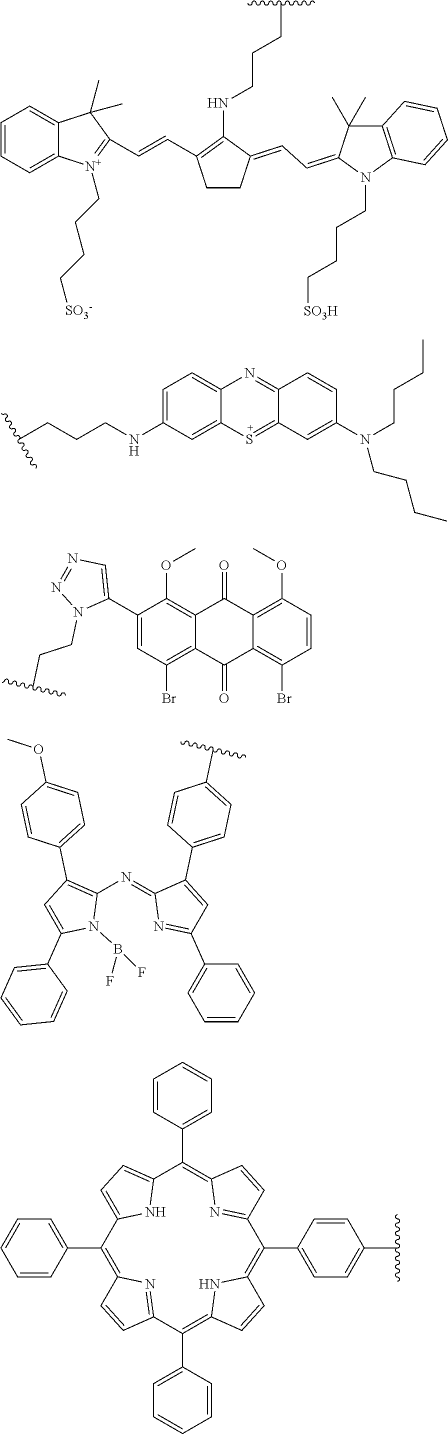

8. Complex according to claim 1, wherein the lanthanide is selected from the group consisting of: Yb, Nd, Ho, Tm, Sm, Dy, Eu, Pr, and Er.

9. Use of a complex according to claim 1 as a fluorescent chromophore.
